(19) United States
(12) Patent Application Publication
(10) Pub. No.: US 2014/0329275 A1
(43) Pub. Date: Nov. 6, 2014

(54) BIOCATALYSIS CELLS AND METHODS

(71) Applicant: REGENTS OF THE UNIVERSITY OF MINNESOTA, Minneapolis, MN (US)

(72) Inventor: Kechun Zhang, Roseville, MN (US)

(21) Appl. No.: 14/350,770

(22) PCT Filed: Oct. 9, 2012
(86) PCT No.: PCT/US2012/059321

§ 371 Date: Apr. 9, 2014

Related U.S. Application Data
(60) Provisional application No. 61/545,328, filed on Oct. 10, 2011.

(51) Int. Cl. C12P 33/00; C12P 7/62

(52) U.S. Cl. CPC C12P 33/00; C12P 7/62
USPC 435/52; 435/254.11; 435/252.3; 435/135

(57) ABSTRACT

In one aspect, the invention provides a recombinant microbial cell modified to exhibit increased biosynthesis of a biosynthetic product compared to a wild-type control. In some cases, the biosynthetic product can be isocaproate; in other cases the biosynthetic product can be isovalerate. In another aspect, the invention provides methods of constructing such recombinant cells. In yet another aspect, the invention provides methods of using the cells to produce the biosynthetic product. In yet another aspect, the invention provides methods of harvesting organic acids from a fermentation culture.

### PRIORITY DATA

This application claims the benefit of U.S. Provisional Application Ser. No. 61/545,328, filed Oct. 10, 2011, which is incorporated by reference herein.

### SUMMARY

In one aspect, the invention provides a recombinant microbial cell modified to exhibit increased biosynthesis of a biosynthetic product compared to a wild-type control. In some cases, the biosynthetic product can be isocaproate; in other cases the biosynthetic product can be isovalerate.

In some embodiments, the recombinant microbial cell can be a fungal cell such as, for example, a member of the Saccharomycetaceae family. In particular embodiments, the fungal cell can be Saccharomyces cerevisiae, Candida rugosa, or Candida albicans.

In other embodiments, the microbial cell can be a bacterial cell such as, for example, a member of the phylum Protobacteria, a member of the phylum Firmicutes, or a member of the phylum Cyanobacteria. In certain of these embodiments, the bacterial cell can be a member of the Enterobacteriaceae family such as, for example, Escherichia coli; a member of the Pseudomonaceae family such as, for example, Pseudomonas putida; a member of the Bacillaceae family such as, for example, Bacillus subtilis; a member of the Streptococcaceae family such as, for example, Lactococcus lactis; or a member of the Clostridiaceae family such as, for example, Clostridium cellulolyticum.

In some embodiments, the recombinant cell can be photosynthetic.

In some embodiments, the recombinant cell can be cellulolytic.

In embodiments in which the biosynthetic product includes isocaproate, the recombinant cell can exhibit an increase in 2-isopropylmalate synthase activity compared to a wild-type control, an increase in ketoleucine elongation activity compared to a wild-type control, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

In embodiments in which the biosynthetic product includes isovalerate, the recombinant cell can exhibit an increase in leucine biosynthesis compared to a wild-type control, an increase in decarboxylase activity compared to a wild-type control, an increase in aldehyde dehydrogenase activity compared to a wild-type control, or an increase in branched-chain ketoacid dehydrogenase activity compared to a wild-type control.

In some embodiments, the recombinant can exhibit a decrease in activity of at least one native ldhA, adhE, pta, pflB, poxB, yqhD enzyme compared to wild-type, thereby decreasing byproduct synthesis compared to a wild-type control.

In another aspect, the invention provides a method that generally includes incubating a recombinant cell modified to exhibit an increase in the biosynthesis of isocaproate in medium that comprises a carbon source under conditions effective for the recombinant cell to produce isocaproate, wherein the carbon source comprises one or more of: glucose, pyruvate, ketovaline, ketoleucine, ketohomoleucine, CO₂, cellulose, xylose, sucrose, arabinose, or glycerol.

In another aspect, the invention provides a method that generally includes incubating a recombinant cell modified to exhibit an increase in the biosynthesis of isovalerate in medium that comprises a carbon source under conditions effective for the recombinant cell to produce isovalerate, wherein the carbon source comprises one or more of: glucose, pyruvate, ketovaline, ketoleucine, isopentanal, CO₂, cellulose, xylose, sucrose, arabinose, or glycerol.

In another aspect, the invention provides a method that generally includes introducing into a host cell a heterologous polynucleotide encoding at least one polypeptide that catalyzes conversion of a carbon source to isocaproate, wherein the at least one polypeptide is operably linked to a promoter so that the modified host cell catalyzes conversion of the carbon source to isocaproate. In some embodiments, the carbon source can include one or more of: glucose, pyruvate, ketovaline, ketoleucine, ketohomoleucine, CO₂, cellulose, xylose, sucrose, arabinose, or glycerol.

In another aspect, the invention provides a method that generally includes introducing into a host cell a heterologous polynucleotide encoding at least one polypeptide that catalyzes conversion of a carbon source to isovalerate, wherein the at least one polypeptide is operably linked to a promoter so that the modified host cell catalyzes conversion of the carbon source to isovalerate. In some embodiments, the carbon source can include one or more of: glucose, pyruvate, ketovaline, ketoleucine, isopentanal, CO₂, cellulose, xylose, sucrose, arabinose, or glycerol. In another aspect, the invention provides a method of harvesting an organic acid from a fermentation broth. Generally, the method includes adjusting the pH of the fermentation broth to about 3.0, adding an organic solvent to the fermentation broth, thereby producing an aqueous phase and a non-aqueous phase, and extracting the organic acid from the aqueous phase. IN some embodiments, the organic solvent can include hexane or oleyl alcohol.

### BRIEF DESCRIPTION OF THE FIGURES

FIG. 1. Reaction pathways to MIAK. a, Current chemical manufacturing starts from acetone and isobutyraldehyde and goes through three reaction steps. b, A renewable approach utilizes engineered E. coli to ferment sugar into isocaproic acid, which then cross-condenses with acetic acid to form MIAK catalyzed by ceria catalysts.

FIG. 2. Biosynthesis of isocaproate. a, Synthetic operons for protein overexpression. b, Screening for aldehyde dehydrogenases that are effective in isocaproate production. c, Kinetic parameters of KDH_{ba} (the most productive enzyme discovered).

FIG. 3. Increase isocaproate production by engineering decarboxylation selectivity. a, Crystal structure of KIVD active site. b, Production level of different strains. i, WT KIVD. F381L/V461AKIVD. iii, F382L/V461AKIVD. iv, M538A/V461AKIVD.v, F542L/V461AKIVD. vi, WT IPDC. vii, V465A IPDC. viii, V540A IPDC. ix, L544A IPDC. c, Sequence alignment of KIVD (SEQ ID NO:83) and Salmonella typhimurium IPDC (SEQ ID NO:84). d, Kinetic parameters of IPDC.

FIG. 4. Increased overexpression of LeuABCD to enhance chain elongation activity. a, Synthetic operons to introduce additional copy of LeuABCD. Wild-type or mutant LeuABCD were encoded on either high or medium copy plasmid. b, Production of isocaproate with different combinations of LeuABCD constructs. i, medium copy mutant leuABCD+high copy WT leuABCD. ii, medium copy mutant leuABCD+high copy mutant leuABCD. iii, medium copy WT leuABCD+high copy mutant leuABCD.

FIG. 5. Plasmid map of p4MV1.

FIG. 6. Plasmid map of p4MV2.

FIG. 7. Reaction pathways to Ketones (e.g., MIBK and DIBK). a, Chemical synthesis starts from petroleum feedstock and involves multiple reaction steps. The process is unsustainable and the overall yield is low. b, “Bio-catalytic conversion” approach utilizes engineered E. coli to ferment renewable sugar into isovaleric acid, which then catalyzed by ceria catalysts (i) cross-condenses with acetic acid to form MIBK and (ii) self-condenses to form DIBK. The process is sustainable and cost-effective. Compounds as follows: diacetone alcohol (1); mesityl oxide (2); 2-hydroxy-2,6-dimethyl-4-heptanone (3); 2,6-dimethyl-2-hepten-4-one (4); isovaleric acid (IVA).

FIG. 8. Engineering E. coli for biosynthesis of isovaleric acid. a, Design a synthetic metabolic pathway (1) by amplifying the leucine biosynthetic pathway to increase the pool of ketoleucine, (2) screening for 2-ketoacid decarboxylases (DC) to decarboxylate ketoleucine into isopentanal, and (3) screening for aldehyde dehydrogenases (DH) to oxidize isopentanal into isovaleric acid. b, Synthetic operons for overexpression of critical proteins. c, Production of isovalerate by different combinations of decarboxylases and dehydrogenases in shake flask. d, Kinetic parameters of the most productive DC and DH. DH1, AldB; DH2, AldH; DH3, KDH_{ba}; DH4, PadA; DC2, KIVD V461A/F381L; DC3, KIVD V461A/F382L; DC4, KIVD V461A/M538A; DC5, KIVD V461A/F542L; DC6, IPDC.

FIG. 9. Production and purification of isovalerate in a 1-L bioreactor. a, Time courses of the fermentation profile. Open symbol denotes AKO1 (BW25113, ΔyqhD) host; solid symbol denotes AKO5 (BW25113, Δpta, ΔpoxB, ΔadhE, ΔldhA, ΔyqhD) host. b, Properties and extraction efficiency of organic solvents. Hexane and oleyl alcohol are suitable for extraction purification of isovaleric acid (b.p. 175° C.).

FIG. 10. Plasmid map of pIVAl.

FIG. 11. Plasmid map of pIVA.

FIG. 12. Active site of KIVD.

FIG. 13. Alternative pathway to isovalerate.

FIG. 14. Plasmid maps of pIBA1 and derivatives pIBA16, pIBA17, and pIBA18.

### DETAILED DESCRIPTION OF ILLUSTRATIVE EMBODIMENTS

Economical biosynthesis of certain ketones can provide commercial and environmental benefits. For example, methyl isoamyl ketone (MIAK) is one of the most widely used solvents (more than 50 million pounds per year), having broad applications in cellulose esters, acrylics and vinyl copolymers (www.epa.gov/oppt/iur/tools/data/2002-vol.html). In particular, MIRK is an excellent solvent for high-solids coatings due to its high solution activity, low density, low surface tension, slow evaporation rate and high boiling point (www.eastman.com/Literature_Center/M/M285.pdf). As another example, industrial ketones such as methyl ethyl ketone (MEK) and methyl isobutyl ketone (MIBK) are commonly used organic solvents in industry. Oxygenated solvents such as aliphatic ketones constitute a large segment of the industrial solvent market. Currently, aliphatic ketones such as methyl isobutyl ketone (MIBK) and diisobutyl ketone (DIBK) cannot be economically obtained from carbohydrates. Here we report genetically modified organisms that may be used for the biosynthesis of such ketones. In certain aspects, we report methods for the biosynthesis of certain ketones. The ketones may be commercial-ready or may be a component of, an ingredient in the production of, or chemical intermediate in the production of, another product.

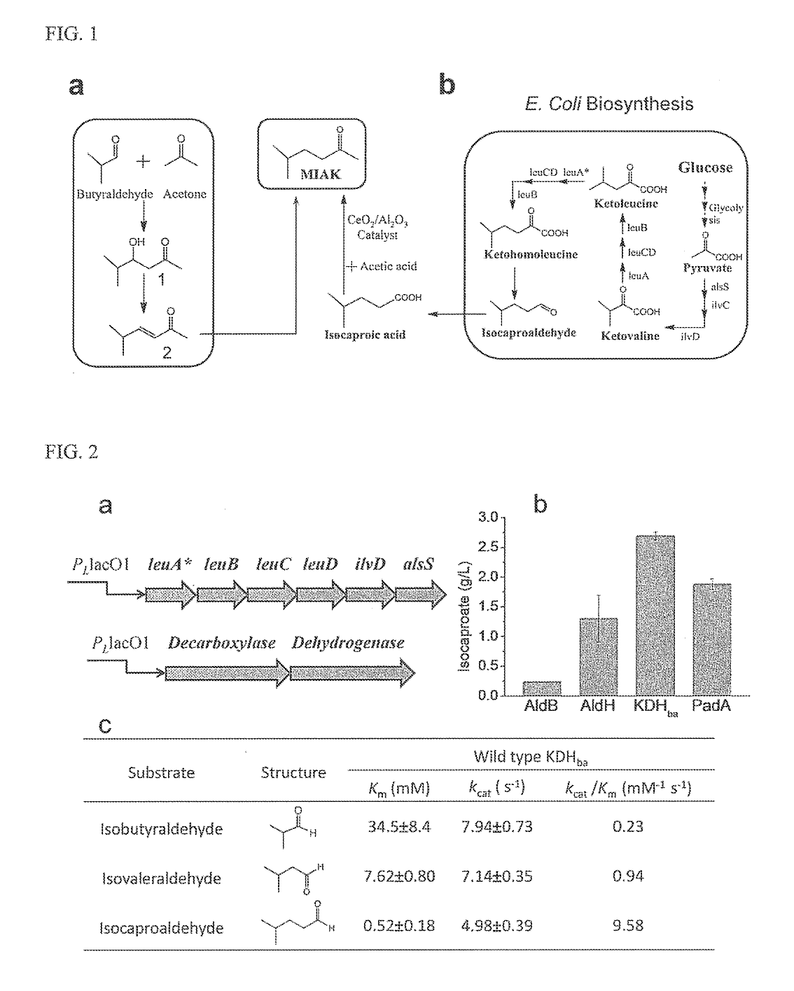

Currently, MIRK is manufactured from isobutyraldehyde and acetone via three chemical reaction steps shown in FIG. 1a: acetone first reacts with isobutyraldehyde to produce 4-hydroxy-5-methyl-2-hexanone 1; 1 is dehydrated to produce 3-ene-5-methyl-2-hexanone 2; the carbon-carbon double bond of unsaturated ketone 2 is selectively hydrogenated to form MIAK.³ These reactions can be carried out using one or two fixed-bed reactors, but it requires high pressure and high temperature. Moreover, the supply of isobutyraldehyde and acetone is derived from the unsustainable petroleum feedstock, whose availability and cost are a serious concern.

To manufacture MIAK in a sustainable fashion, we have developed a hybrid biocatalytic conversion approach (FIG. lb) by combining both synthetic biology and chemical synthesis. Based on retrosynthesis analysis, cross-condensation of isocaproic acid with acetic acid to MIAK represents an excellent synthetic route. With the release of only carbon dioxide and water, the one-step ketonization is an environmentally friendly and highly efficient method. To synthesize MIAK, isocaproate is preferably obtained by microbial fermentation from renewable carbon sources such as, for example, glucose. However, no organisms to date have been discovered to accumulate a significant amount of isocaproate although it is a minor metabolite in Clostridium difficile (Kim et al., Appl. Environ. Microbiol. 72:6062-6069 (2006)).

In one aspect of this work, an isocaproate overproducer was created via three steps, by (1) engineering enzymes to generate ketohomoleucine through elongating ketoleucine with one more carbon, (2) screening for optimal ketoacid decarboxylases that convert ketohomoleucine to isocaproaldehyde, and (3) screening for active aldehyde dehydrogenases that oxidize isocaproaldehyde into isocaproate. In some exemplary embodiments, E. coli may be chosen as the host. E. coli naturally can convert glucose to ketoleucine via the branched-chain amino acid biosynthetic pathway (Atsumi et al., Nature 451:86-9 (2008)). E. coli carrying a certain leuA mutation can produce ketohomoleucine (Zhang et al., Proc Natl Acad Sci USA 105:20653-8 (2008)), but no E. coli thus described can biosynthesize isocaproate. Herein we describe building a synthetic metabolic pathway to achieve high-level production of isocaproate directly from glucose. The isocaproate may subsequently be condensed with acetic acid into MIAK with high yield and selectivity (FIG. 1b).

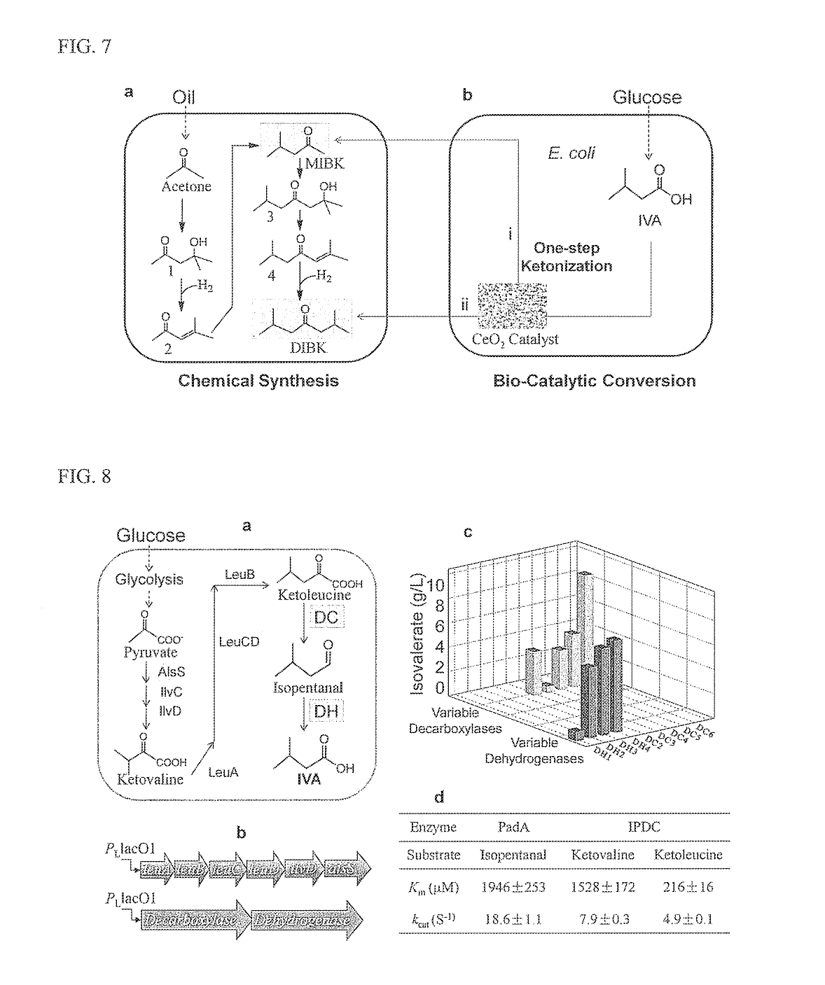

Similarly, at present, the manufacturing processes of aliphatic ketones can be relatively complicated. As illustrated in FIG. 7, the petroleum-derived raw material acetone is chemically processed to MIBK via three reaction steps: (i) aldol condensation to diacetone alcohol 1, (ii) dehydration to mesityl oxide 2 and (iii) selective hydrogenation of the unsaturated ketone 2. MIBK can further react with another acetone molecule to yield diisobutyl ketone (DIBK) following the three-step process. Although multifunctional catalysts have been developed to enable these reaction series in one vessel, the yield to MIBK or DIBK can be limited to only around 30% or 10% (Chen et al., Appl. Catal. A: Gen. 169:207-214 (1998)). In addition, the operation requires high pressure (10˜100 atm), and the supply of acetone is dependent on the availability of petroleum feedstock.

In another aspect of this work, we present a general strategy for synthesizing aliphatic ketones from carbohydrates. This biocatalytic conversion approach exploits engineered biosynthesis to produce ketones that may serve as the starting material for further chemical processes (FIG. 7b). Single-step ketonization, in which two molecules of carboxylic acid couple to form a ketone, is environmentally friendly (no waste generation) and highly efficient. Another attractive feature of the approach is that carboxylic acids could be obtained by microbial fermentation. As one example, to synthesize MIBK and DIBK, we have engineered an E. coli overproducer to convert glucose into isovaleric acid.

We have engineered E. coli to overproduce isovalerate by (1) amplifying the leucine biosynthetic pathway to increase the pool of ketoleucine, (2) screening for 2-ketoacid decarboxylases that convert ketoleucine to isopentanal, and (3) screening for aldehyde dehydrogenases that oxidize isopentanal into isovalerate (FIG. 8a). The first step relies on genetic manipulation of the branched-chain amino acid metabolic pathway. In this pathway, the condensation of two pyruvate molecules leads to ketovaline after a series of biochemical reactions catalyzed by acetolactate synthase (IlvIH), ketol-acid reductoisomerase (IlvC) and dihydroxy-acid dehydratase (IlvD). Ketovaline is then elongated to ketoleucine enabled by 2-isopropylmalate synthase (LeuA), isopropylmalate isomerase complex (LeuCD) and isopropylmalate dehydrogenase (LeuB). Generally, amino acid pathways are subjected to strict metabolic regulation. In E. coli, IlvIH and LeuA are sensitive to feedback inhibition by valine and leucine (Calvo et al., J Bacteriol. 97:1272 (1969)). To disable such regulatory mechanisms, we introduced the degradative acetolactate synthase (AlsS) from Bacillus subtilis, which is not inhibited by valine and has a much higher catalytic activity compared to its E. coli counterpart IlvIH (Atsumi et al., Nature 451:86-89 (2008)); we also used a 2-isopropylmalate synthase (LeuA) with the G462D mutation (Zhang et al., Proc. Natl. Acad. Sci. USA 105:20653-20658 (2008)) to prevent the regulatory binding of leucine. The corresponding genes encoding LeuA, LeuB, LeuC, LeuD, IlvD, and AlsS were sequentially assembled into a single operon under the control of the P_{L}lacO1 promoter on a medium copy plasmid (FIG. 8b).

In the description of exemplary embodiments that follow, certain metabolic enzymes—and the natural source of those enzymes—are specified. These are merely examples of suitable enzymes and suitable sources of the specified enzymes. Alternative enzymes with similar catalytic activities are possible, as are homologs that are obtainable from different microbial species or strains. Accordingly, the exemplary embodiments described herein should not be construed as limiting the scope of the microbes or methods that are reflected in the claims.

### Isocaproate Biosynthesis

As shown in FIG. 1b, during biosynthesis of branched-chain amino acids, two pyruvate molecules may be condensed to form ketovaline after consecutive biochemical reactions catalyzed by acetolactate synthase (IlvIH), ketol-acid reductoisomerase (IlvC) and dihydroxy-acid dehydratase (IlvD). Since the degradative acetolactate synthase (AlsS) of Bacillus subtilis is not inhibited by valine and has much higher catalytic activity compared to its E. coli counterpart IlvIH (Atsumi et al., Nature 451:86-9 (2008)), we used AlsS for the synthetic pathway. Ketovaline may then be elongated to ketoleucine, enabled by 2-isopropylmalate synthase (LeuA), isopropylmalate isomerase complex (LeuCD), and isopropylmalate dehydrogenase (LeuB). A LeuA mutant (G462D/S139G/H97L, LeuA*) was reported to enable the elongation of ketoleucine to ketohomoleucine (Zhang et al., Proc Natl Acad Sci USA 105:20653-8 (2008)). Thus, we constructed a synthetic operon (FIG. 2a) under the control of the P_{L}lacO1 promoter as a first part of the synthetic metabolic pathway to drive carbon flux towards ketohomoleucine. This operon is composed of six genes on a medium copy plasmid in the transcriptional order thrA- leuA*-leuB-leuC-leuD-ilvD-alsS. In the Erhlich pathway, 2-keto acids are converted to aldehydes and then to alcohols (Hazelwood et al., Appl. Environ. Microbiol. 74:2259-2266 (2008)). To change the products from alcohols to acids, we introduced a non-natural metabolic step to oxidize rather than reduce the aldehydes. This step involves the function of aldehyde dehydrogenase. Therefore, we constructed another operon that encodes different 2-ketoacid decarboxylases and aldehyde dehydrogenases (FIG. 2a).

### Screening Aldehyde Dehydrogenases for Isocaproate Production

We chose several aldehyde dehydrogenases that were reported to possess decent activity towards aliphatic aldehydes as candidate enzymes (Zhang et al., ChemSusChem 4:1068-1070 (2011)). They are E. coli acetaldehyde dehydrogenase AldB (Ho and Weiner, J. Bacteriol. 187:1067-1073 (2005)), E. coli 3-hydroxypropionaldehyde dehydrogenase AldH (Jo et al., Appl. Microbiol. Biotechnol. 81:51-60 (2008)), E. coli phenylacetaldehyde dehydrogenase PadA (Rodriguez Zavala et al., Protein Sci. 15:1387-1396 (2006)), and Burkholderia ambifaria α-ketoglutaric semialdehyde dehydrogenase KDH_{ba} (Watanabe et al., J. Biol. Chem. 282:6685-6695 (2007)). These enzymes (designated DH) were individually cloned after 2-ketoacid decarboxylase (KIVD) from Lactococcus lactis (de la Plaza et al., FEMS Microbiol. Lett. 238:367-74 (2004)).

The BW25113 E. coli strain with yqhD gene deletion (Atsumi et al., Appl. Microbiol. Biotechnol. 85:651-657 (2010)) was used as the fermentation host in order to decrease the reduction activity of endogenous alcohol dehydrogenases. After transformation with the two constructed plasmids, shake flask femientation was performed in M9 minimal medium containing 40 g/L glucose at 30° C. Fermentation broths after 48 hours were analyzed by HPLC. As seen in FIG. 2b, the selected aldehyde dehydrogenases could function in producing isocaproate even though they have different activities. AIdB produced only 0.24 g/L isocaproate, but AldH, PadA and KDH_{ba} increased the production level to 1.30 g/L, 1.88 g/L and 2.69 g/L. The best enzyme KDH_{ba} was purified and characterized (FIG. 2c). The Michaelis-Menten constant (Kₘ) of KDH_{ba} decreased as the size of the substrates increased: towards isobutyraldehyde, Kₘ is 34.5 mM; and the number is 0.52 mM for the bulkier compound isocaproaldehyde. On the other hand, the catalytic rate constant (kₐₜ) is similar for all three aldehydes. Therefore, the specificity constant k_{cat}/Kₘ of KDH_{ba} towards isocaproaldehyde is 10-fold higher and 42-fold higher than those towards isovaleraldehyde and isobutyraldehyde, respectively, which could explain why KDH_{ba} was the best enzyme discovered to synthesize isocaproate instead of isobutyrate and isovalerate (Table 2).

### Engineering Decarboxylases to Increase Product Selectivity

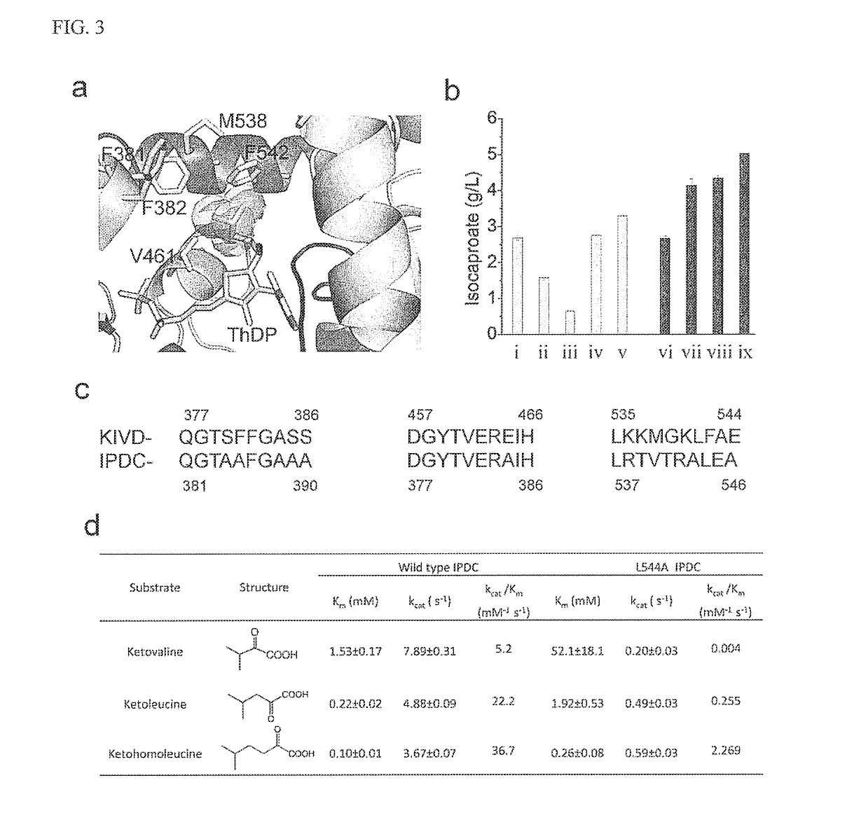

While the discovery of KDH_{ba} has maximized the selectivity in the oxidation step, the promiscuous nature of KIVD decarboxylase may not necessarily confer optimal selectivity in the decarboxylation step. To increase the production of isocaproate and decrease the formation of byproducts, we investigated the effect of enlarging the binding pocket of KIVD. According to the crystal structure (PDB: 2VBG), amino acid residues Phe-381, Phe-382, Val-461, Met 538, and Phe-542, in combination with the cofactor thiamine diphosphate (ThDP), delineate the active site of KIVD (FIG. 3a) (Berthold et al., Acta. Crystallogr. D. Biol. Crystallogr. 63:1217-1224 (2007)). Mutation V461A was reported to decrease the activity of KIVD towards smaller substrates (Zhang et al., Proc Natl Acad Sci USA 105:20653-8 (2008)). To further increase the selectivity towards a larger substrate, four additional mutations (F381L, F382L, M538A or F542L) were created in combination with V461A to further increase the size of the KIVD active site. The fermentation data indicated that the M538A/V461A double mutant and the F542L/V461A double mutant exhibited enhanced isocaproate production (FIG. 3b, i-v). Compared to wild-type KIVD, for example, the F542L/V461A mutant increased isocaproate production by 23% to 3.30 g/L. Nevertheless, the yield of 4MV was only 25.6% of the theoretical maximum.

We investigated the effect of further engineering another enzyme, indolepyruvate decarboxylase (IPDC) from Salmonella typhimurium, into our biosynthetic system. Since E. coli and S. typhimurium diverged from a common ancestor, S. typhimurium enzymes should express well in E. coli. While E. coli genome does not encode any 2-ketoacid decarboxylase, S. typhimurium has IPDC even though its activity was not fully investigated. Interestingly, the combination of wild-type IPDC and KDH_{ba} produced 2.67 g/L isocaproate (FIG. 3b, vi) from 40 g/L glucose, which is almost the same as that (2.69 g/L) produced from wild-type KIVD. Based on the protein sequence alignment of [PDC and KIVD, Ala-385, Phe-386, Val-465, Val-540 and Leu-544 are the corresponding active site residues of IPDC (FIG. 3c). Since V461A, M538A and F542L mutations in KIVD were helpful in increasing isocaproate production, single site mutations V465A, V540A and L544A were created in S. typhimurium IPDC. Fermentation results demonstrated that V465A, V540A and L544A IPDC mutants produced 4.14 g/L, 4.35 g/L, and 5.02 g/L isocaproate, respectively (FIG. 3b). Thus, the production by L544A IPDC was 52% higher than that produced by F542L/V461A KIVD, and 88% higher than that produced by wild-type IPDC. These results suggested that F542 of KIVD or L544 of IPDC is involved in determining the substrate selectivity towards ketohomoleucine. For wild-type IPDC, the selectivity constant (kcat/Km) towards ketoleucine and ketohomoleucine are 22.2 and 36.7 mM⁻¹ s⁻¹ (FIG. 3d), respectively, indicating that wild-type IPDC has similar selectivity for the C6 and C7 ketoacids. However, the L544A mutation significantly increases the selectivity towards the C7 ketoacid ketohomoleucine, which is 10-fold higher than the selectivity towards ketoleucine and 567-fold higher than the selectivity towards ketovaline. The consequence is L544A mutant produced the largest amount of isocaproate while the production of derivatives of ketovaline and ketoleucine were reduced (Table 2).

### Maximizing Carbon Flux by Increasing Overexpression of Pathway Enzymes

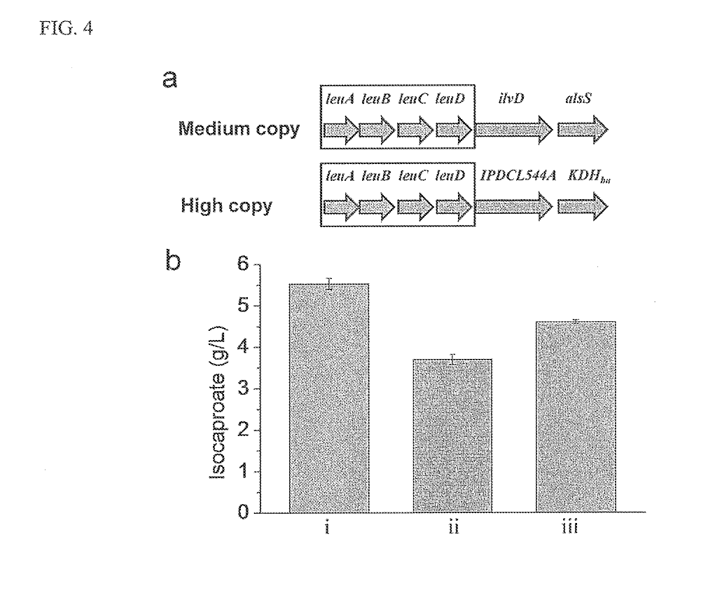

While engineering IPDC improved isocaproate production, there were still some byproducts such as isobutyrate, isobutanol, isovalerate, and isopentanol accumulated in the fermentation broth. Since these byproducts were generated from decarboxylation of ketovaline and ketoleucine, we investigated increasing the chain elongation activity of LeuABCD to maximize the carbon conversion from ketovaline and ketoleucine to ketohomoleucine. An additional copy of wild-type or mutant LeuABCD encoded on either high- or medium- copy plasmid was introduced (FIG. 4a). Fermentation results demonstrated that mutant leuABCD on a medium copy plasmid plus wild-type leuACD on a high copy plasmid generated 5.53 g/L isocaproate (FIG. 4b, i), which increased 10.2% compared to that in the strain carrying only one copy of mutant leuABCD on a medium copy plasmid. The other two combinations (medium copy mutant leuABCD+high copy mutant leuABCD, or medium copy WT leuABCD+high copy mutant leuABCD) did not perform as well as the first combination.

### Isovalerate Biosynthesis

Thus far no microorganism has been discovered to accumulate a significant quantity of isovalerate, although it is a minor metabolite in lactobacillus and yeast (<3 mg/L) (Hazelwood et al., Appl. Environ. Microbiol. 74:2259-2266 (2008; Lambrechts et al., S. Afr. J. Enol. Vitic. 21:97-129 (2000)). As briefly stated above, we have engineered E. coli to overproduce isovalerate. In the Erhlich pathway, 2-keto acids are normally processed to aldehydes by 2-ketoacid decarboxylases, and then to alcohols by alcohol dehydrogenases (Hazelwood et al., Appl. Environ. Microbiol. 74:2259-2266 (2008)). To shift the products from alcohols to acids, we constructed a non-natural metabolic pathway to oxidize rather than reduce aldehydes. We chose several aldehyde dehydrogenases as candidate enzymes: E. coli acetaldehyde dehydrogenase AldB; E. coli 3-hydroxypropionaldehyde dehydrogenase AldH; E. coli phenylacetaldehyde dehydrogenase PadA; and Burkholderia ambifaria α-ketoglutaric semialdehyde dehydrogenase KDH_{ba} (Zhang et al., ChemSusChem 4:1068-1070 (2011)). These enzymes (designated DH) were individually cloned after 2-ketoacid decarboxylase (KIVD) from Lactococcus lactis (de la Plaza et al., FEMS Microbiol. Lett. 238, 367-74 (2004)) to build an expression cassette on a high copy plasmid (FIG. 8b). The two constructed plasmids were then transformed into an E. coli strain AKO1 (BW25113, ΔyqhD) (Zhang et al., ChemSusChem 4:1068-1070 (2011)). Shake flask fermentation was performed at 30° C. for 48 hours in M9 minimal medium containing 40 g/L glucose. The choice of aldehyde dehydrogenases affected the fermentation outcome (DH1-4, FIG. 8c). AldB could only produce 0.8 g/L isovalerate, which suggests that its activity towards isopentanal is low. In comparison, AldH, KDH_{ba} and PadA significantly increased the production level to 5.8 g/L, 7.1 g/L and 7.5 g/L. It is interesting to discover that these enzymes are promiscuous enough to catalyze the oxidation of isopentanal, although these enzymes’ natural functions do not include isovalerate biosynthesis. PadA was purified and assayed in vitro to characterize its enzymatic activity (FIG. 8d). The Michaelis-Menten constant (Kₘ) and the catalytic rate constant (k_{cat}) of PadA for isopentanal were determined to be 1946 μM and 18.6 s⁻¹. Therefore, PadA possesses strong catalytic activity towards the noncognate substrate isopentanal.

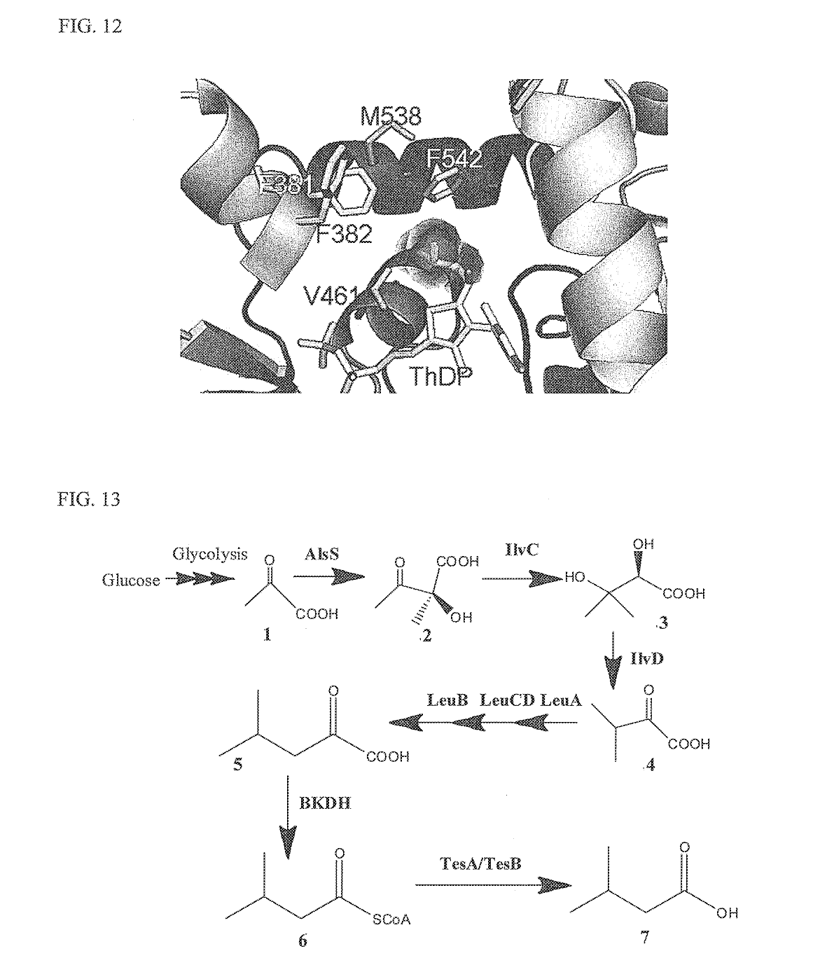

Byproduct isobutyrate was present in the fermentation broth at a concentration of approximately 0.7 g/L. This may have been a result KIVD decarboxylating the intermediate metabolite ketovaline as well as ketoleucine. In order to achieve higher selectivity towards ketoleucine, we examined the effect of enlarging the binding pocket of KIVD. Based on the crystal structure, the active site residue V461 was mutated to alanine (Zhang et al., Proc. Natl. Acad. Sci. USA 105:20653-20658 (2008)). A second mutation—either F381L, F382L, M538A, or F542L—was introduced in addition to the V461 mutation (FIG. 12). These mutants reduced the isobutyrate concentration to a range of 0.1 to 0.2 g/L during fermentation (Table 4). The V461A/F542L mutant generated 4.3 g/L isovalerate.

We next investigated cloning another enzyme, indolepyruvate decarboxylase (IPDC) from Salmonella typhimurium. To our surprise, the combination of IPDC and PadA produced 8.9 g/L isovalerate (DC 6, FIG. 8c) from 40 g/L glucose, which represents a yield of 0.22 g/ g glucose that is 58% of the theoretical maximum. Enzymatic assay (FIG. 8d) indicated that, compared to KIVD (Yep et al., Bioorg. Chem. 34:325-336 (2006)), for the smaller substrate ketovaline, IPDC has a significantly lower k_{cat} (7.9 s⁻¹ versus 48 s⁻¹) and a close Kₘ (1528 μM versus 2800 μM); for ketoleucine, IPDC still has a lower k_{cat} (4.9 s⁻¹ versus 49 s⁻¹), but a much lower Kₘ (216 μM versus 37000 μM). Thus, the specificity constant k_{cat}/Kₘ of IPDC towards ketoleucine is 4-fold higher than that towards ketovaline, which could explain the better performance of IPDC over KIVD with respect to isovalerate production.

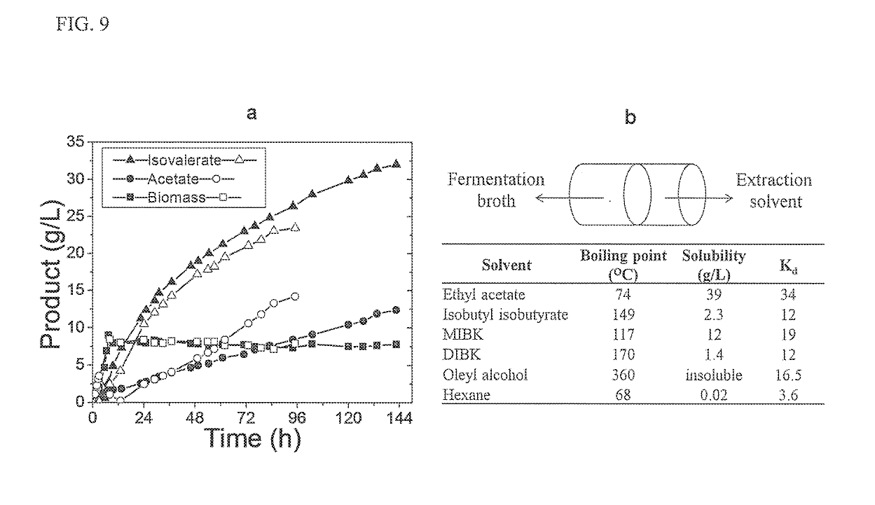

To explore the possibility of scale-up production, we translated the fermentation process from shake flasks to a 1-liter bioreactor (FIG. 9a). The culture produced 23.4 g/L isovalerate in 95 hours. However, the production did not proceed beyond this time point and acetate accumulated to 14.2 g/L. To reduce acetate formation (Causey et al., Proc. Natl. Acad. Sci. USA 100:825-832 (2003)), we constructed a new E. coli strain AKO5 (BW25113, Δpta, ΔpoxB, ΔadhE, ΔldhA, ΔyqhD) as the production host. As seen in FIG. 9a, while AKO1 and AKO5 had a similar growth profile in the fermenter, the rate of acetate accumulation in AKO5 was significantly decreased and the rate of isovalerate production was improved. The fmal titer of isovalerate reached 32 g/L after 142 hours. Thus, isovalerate production in the engineered E. coli strain was increased by over 10,000 times compared to that in natural organisms (Lambrechts et al., S. Afr. J. Enol. Vitic. 21:97-129 (2000)).

We also explored biosynthetic alternatives for producing isovalerate. One alternative non-natural biosynthetic pathway was achieved by overexpressing a metabolic pathway from pyruvate (FIG. 13, Compound 1) to isovalerate (FIG. 13, Compound 7). This novel, non-natural pathway begins with the condensation of two pyruvate molecules to generate ketovaline (FIG. 13, Compound 4), catalyzed by an acetolactate synthase (AlsS), an acetohydroxy acid isomeroreductase (IlvC), and a dihydroxy-acid dehydratase (IlvD). We used these enzymes from Bacillus subtilis, but homologues from other microbial species may be just as suitable. The ketovaline is then converted into a pool of ketoleucine (FIG. 13, Compound 5) using the leucine synthesis pathway enzymes 2-isopropylmalate synthase (LeuA), 3-isopropylmalate dehydrogenase (LeuB), and isopropylmalate isomerase (LeuC and LeuD). Pseudomonas putida branched-chain ketoacid dehydrogenase (BKDH) then converts the ketoleucine to isovaleryl-CoA (FIG. 13, Compound 6), and E. coli thioesterase II (TesB) completes the pathway to isovaleric acid. E. coli engineered in this way produced 10.27 g/L isovalerate. (Table 5, Example 3).

Since isovalerate is protonated under acidic conditions and becomes hydrophobic, we exploited this feature to develop a purification method. The pH of the fermentation broth was adjusted to 3.0, and then solvents were applied to extract isovaleric acid out of the aqueous phase (FIG. 9b). Among the six solvents tested, hexane and oleyl alcohol appear to be suitable for the extraction purpose: isovaleric acid has a high distribution coefficient K_{d} in both solvents; the solvents are water insoluble (minimal loss during extraction); the boiling points of both solvents are very different from that of isovaleric acid (facile separation through distillation).

Isocaproate produced as described herein may be used as a raw material for the production of industrial chemicals such as, for example, MIAK. Isovalerate herein may be used as a raw material for the production of industrial chemicals such as, for example, MIBK and/or DIBK. The cells and methods described herein may therefore be used to provide renewable sources of raw materials for the production of industrial chemicals in an economical and environmentally advantageous manner.

This research opens a new path for the production of renewable chemicals by exploiting certain benefits of microbial fermentation. The process and raw material requirements are simplified compared to the current chemical manufacturing process. While this approach can be easily extended to other aliphatic ketones, we envision that the concept has broader implications. Currently, to produce industrial chemicals such as plastics, fertilizers, and pharmaceuticals, 99% of the feedstock materials comes from petroleum or natural gas (McFarlane and Robinson, Survey of Alternative Feedstocks for Commodity Chemical Manufacturing, available on the World Wide Web at info.ornl.gov/sites/publications/files/Pub8760.pdf (2007)). The existing chemical synthesis routes are optimized based on the availability and cost of petroleum stock. The cells and methods described herein allow the design of more simplified manufacturing routes based on chemical precursors that can be efficiently biosynthesized from renewable carbohydrates.

Thus, in one aspect, the invention provides recombinant microbial cell modified to exhibit increased biosynthesis of isocaproate compared to a wild-type control. In another aspect, the invention provides a recombinant microbial cell modified to exhibit increased biosynthesis of isovalerate compared to a wild-type control. In some cases, the wild-type control may be unable to produce isocaproate or isovalerate and, therefore, an increase in the biosynthesis of a particular product may reflect any measurable biosynthesis of that product. In certain embodiments, an increase in the biosynthesis of an isocaproate or isovalerate can include biosynthesis sufficient for a culture of the microbial cell to accumulate isocaproate or isovalerate to a predetermine concentration.

The predetermined concentration may be any predetermined concentration of the product suitable for a given application. Thus, a predetermined concentration may be, for example, a concentration of at least 0.1 g/L such as, for example, at least 0.5 g/L, at least 1.0 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, at least 10 g/L, at least 20 g/L, at least 50 g/L, at least 100 g/L, or at least 200 g/L.

The recombinant cell can be, or be derived from, any suitable microbe including, for example, a prokaryotic microbe or a eukaryotic microbe. As used herein, the term “or derived from” in connection with a microbe simply allows for the “host cell” to possess one or more genetic modifications before being modified' to exhibit the indicated increased biosynthetic activity. Thus, the term “recombinant cell” encompasses a “host cell” that may contain nucleic acid material from more than one species before being modified to exhibit the indicated biosynthetic activity.

In some embodiments, the host cell may be selected to possess one or more natural physiological activities. For example, the host cell may be photosynthetic (e.g., cyanobacteria) or may be cellulolytic (e.g., Clostridium cellulolyticum).

In some embodiments, the recombinant cell may be, or be derived from, a eukaryotic microbe such as, for example, a fungal cell. In some of these embodiments, the fungal cell may be, or be derived from, a member of the Saccharomycetaceae family such as, for example, Saccharomyces cerevisiae, Candida rugosa, or Candida albicans.

In other embodiments, the recombinant cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis), members of the Clostridiaceae family (e.g., Clostridium cellulolyticum) and, for example, members of the Streptococcaceae family (e.g., Lactococcus lactis). In other cases, the bacterium may be a member of the phylum Cyanobacteria.

In some embodiments, the increased biosynthesis of isocaproate compared to a wild-type control can include an increase in elongating ketoleucine to ketohomoleucine compared to a wild-type control, an increase in ketoacid decarboxylase activity compared to a wild-type control, and/or an increase in aldehyde dehydrogenase activity compared to a wild-type control. In other embodiments, the increased biosynthesis of isocaproate compared to a wild-type control can include an increase in conversion of ketohomoleucine to isocaproaldehyde compared to a wild-type control and/or an increase in conversion of isocaproaldehyde to isocaproate compared to a wild-type control. In some cases, at least a portion of the increase in ketoacid decarboxylase activity can result from modification of the ketoacid decarboxylase enzyme. For example, 2-ketoacid decarboxylase of Lactococcus lactis (or an analog) may be modified to include at least one amino acid substitution selected from: V461A, M538A, or F542L, or an analogous substitution. In some cases, the 2-ketoacid decarboxylase can be modified to include the V461A substitution (or an analogous substitution) in combination with either the M528A substitution (or an analogous substitution) or the V461A substitution (or an analogous substitution).

As used herein, the term “analog” refers to a related enzyme from the same or a different microbial source with similar enzymatic activity. As such, analogs often show significant conservation, it is a trivial matter for a person of ordinary skill in the art to identify a suitably related analog. Also, it is a trivial matter for a person of ordinary skill in the art to identify an “analogous substitution” by aligning the amino acid sequence of the analog with the amino acid sequence of the reference enzyme. Thus, positional differences and/or amino acid residue differences may exist between the recited substitution and an analogous substitution despite conservation between the analog and the reference enzyme. In other embodiments, the increased biosynthesis of isovalerate compared to a wild-type control comprises increased leucine biosynthesis compared to a wild-type control, increased decarboxylase activity compared to a wild-type control, or increased aldehyde dehydrogenase activity compared to a wild-type control.

In some embodiments, the recombinant cell can exhibit an increase in indolepyruvate decarboxylase (IPDC) activity. The increase in IPDC activity can result from expression of an IPDC enzyme. Exemplary IPDC enzymes include, for example, any one of the polypeptides reflected in any one of SEQ ID NO:1-21. Thus, in some embodiments, the recombinant cell can include a heterologous nucleotide sequence that encodes an IPDC decarboxylase such as, for example, any one of the polypeptides reflected in any one SEQ ID NO:1-21.

In some embodiments the recombinant cell can exhibit an increase in aldehyde dehydrogenase activity. The increase in aldehyde dehydrogenase activity can result from expression of an aldehyde dehydrogenase enzyme. Exemplary aldehyde dehydrogenase enzymes include, for example, any one of the polypeptides reflected in any one of SEQ ID NO:22-55. Thus, in some embodiments, the recombinant cell can include a heterologous nucleotide sequence that encodes an aldehyde dehydrogenase such as, for example, any one of the polypeptides reflected in any one SEQ ID NO:22-55.

In some embodiments, the recombinant cell can exhibit an increase in branched-chain ketoacid dehydrogenase (BKDH) activity. The increase in branched-chain ketoacid dehydrogenase activity can result from expression of a branched-chain ketoacid dehydrogenase enzyme. Exemplary branched-chain ketoacid dehydrogenase enzymes include, for example, any one of the polypeptides reflected in any one of SEQ ID NO: 91-93. Thus, in some embodiments, the recombinant cell can include a heterologous nucleotide sequence that encodes a branched-chain ketoacid dehydrogenase enzyme such as, for example, any one of the polypeptides reflected in any one SEQ ID NO:91-93.

In some embodiments, the recombinant cell can include a heterologous nucleotide sequence that encodes a variant of E. coli 2-isopropylmalate synthase that exhibits reduced enzymatic activity compared to wild-type E. coli 2-isopropylmalate synthase. In some cases, the modified E. coli 2-isopropylmalate synthase includes a G462D amino acid substitution or an analogous amino acid substitution.

As used herein, the terms “activity” with regard to particular enzyme refers to the ability of a polypeptide, regardless of its common name or native function, to catalyze the conversion of the enzyme's substrate to a product, regardless of whether the “activity” as less than, equal to, or greater than the native activity of the identified enzyme. Methods for measuring the biosynthetic activities of cells are routine and well known to those of ordinary skill in the art.

As used herein, an increase in catalytic activity can be quantitatively measured and described as a percentage of the catalytic activity of an appropriate wild-type control. The catalytic activity exhibited by a genetically-modified polypeptide can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild-type control.

Alternatively, an increase in catalytic activity may be expressed as at an increase in k_{cat} such as, for example, at least a two-fold increase, at least a three-fold increase, at least a four-fold increase, at least a five-fold increase, at least a six-fold increase, at least a seven-fold increase, at least an eight-fold increase, at least a nine-fold increase, at least a 10-fold increase, at least a 15-fold increase, or at least a 20-fold increase in the k_{cat} value of the enzymatic conversion.

An increase in catalytic activity also may be expressed in terms of a decrease in Kₘ such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the Kₘ value of the enzymatic conversion.

A decrease in catalytic activity can be quantitatively measured and described as a percentage of the catalytic activity of an appropriate wild-type control. The catalytic activity exhibited by a genetically-modified polypeptide can be, for example, no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, no more than 1% of the activity, or 0% of the activity of a suitable wild-type control.

Alternatively, a decrease in catalytic activity can be expressed as an appropriate change in a catalytic constant. For example, a decrease in catalytic activity may be expressed as at a decrease in k_{cat} such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the kₐₜ value of the enzymatic conversion.

A decrease in catalytic activity also may be expressed in terms of an increase in Kₘ such as, for example, an increase in Kₘ of at least two-fold, at least three-fold, at least four-fold, at least five-fold, at least six-fold, at least seven-fold, at least an eight-fold, at least nine-fold, at least 10-fold, at least 15-fold, at least 20-fold, at least 25-fold, at least 30-fold, at least 35-fold, at least 40-fold, at least 45-fold, at least 50-fold, at least 75-fold, at least 100-fold, at least 150-fold, at least 200-fold, at least 230-fold, at least 250-fold, at least 300-fold, at least 350-fold, or at least 400-fold.

Thus, in another aspect, we describe herein methods for biosynthesis of isocaproate or isovalerate. Generally, the methods includes incubating a recombinant cell as described herein in medium that includes a carbon source under conditions effective for the recombinant cell to produce isocaproate or isovalerate. For producing isocaproate, the carbon source can include one or more of: glucose, pyruvate, ketovaline, ketoleucine, or ketohomoleucine. For producing isovalerate, the carbon source can include one or more of: glucose, pyruvate, ketovaline, ketoleucine, or isopentanal. In addition, the carbon sources for cell growth can be CO₂, cellulose, glucose, xylose, sucrose, arabinose, glycerol, etc. as long as the related carbon assimilation pathways are introduced in the engineered microbe.

In yet another aspect, we describe herein methods for introducing a heterologous polynucleotide into cell so that the host cell exhibits an increased ability to convert a carbon source to isocaproate or isovalerate. For cells to produce isocaproate, the heterologous polynucleotide can encode a polypeptide operably linked to a promoter so that modified cell catalyzes conversion of the carbon source to isocaproate. In some of these embodiments, the carbon source can include one or more of glucose, pyruvate, ketovaline, ketoleucine, or ketohomoleucine. For cells to produce isovalerate, the heterologous polynucleotide can encode a polypeptide operably linked to a promoter so that modified cell catalyzes conversion of the carbon source to isovalerate. In some of these embodiments, the carbon source can include one or more of glucose, pyruvate, ketovaline, ketoleucine, or isopentanal. The host cells for such methods can include, for example, any of the microbial species identified above with regard to the recombinant cells described herein.

In some cases, the recombinant cell can include a genetically-modified version of a polypeptide that catalyzes the conversion of isobutyraldehyde to isobutanol. An exemplary polypeptide of this type can include, for example, can be a genetically-modified version of an alcohol dehydrogenase such as, for example, a polypeptide encoded by a genetically-modified adhE or a genetically-modified adhP. In other embodiments, the genetically-modified polypeptide can be genetically-modified version of an ethanolamine utilization protein such as, for example, a polypeptide encoded by a genetically-modified eutG. In some embodiments, the genetically-modified polypeptide can be a polypeptide encoded by a genetically-modified yiaY, a genetically-modified yqhD, or a genetically-modified yigB.

As used in the preceding description, the term “and/or” means one or all of the listed elements or a combination of any two or more of the listed elements; the term “comprises” and variations thereof do not have a limiting meaning where these terms appear in the description and claims; nnless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is illustrated by the following example. It is to be understood that the particular examples, materials, amounts, and procedures are to be interpreted broadly in accordance with the scope and spirit of the invention as set forth herein.

### EXAMPLES

### Example 1

### 1. Vector Construction

The E. coli strain XL10-gold (Stratagene) was used as the host to conduct all cloning procedures. All the primers (Table 1) were purchased from Eurofms MWG Operon. PCR amplification were carried out with using Phusion® High-Fidelity DNA polymerase (New England Biolabs; Ipswich, Mass.). All the plasmids were sequenced to make sure their corrections.

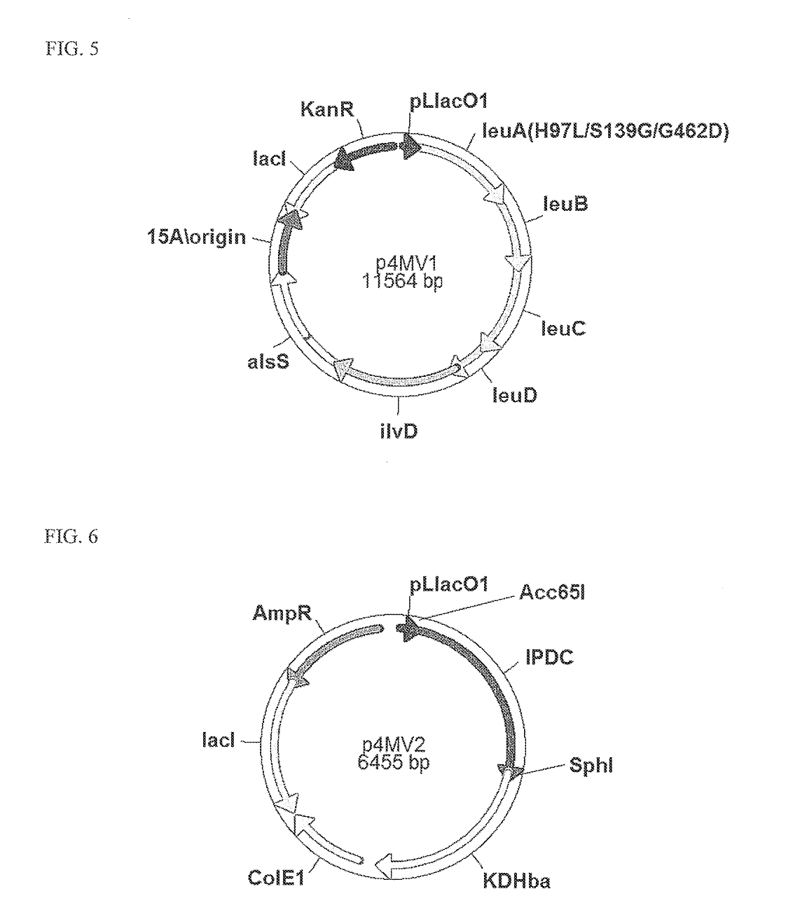

The 4702 by fragment of leuABCD operon, in which leuA carries G462D/H97L/S139G mutations, was obtained from pZE_LeuABCDKA6 (Zhang et al., Proc Natl Acad Sci USA 105:20653-8 (2008)) plasmid after Acc65I digestion, and inserted into the Acc65I site of pZAlac_ilvD_alsS (pIBA1) (Zhang et al., ChemSusChem 4:1068-1070 (2011)) to create p4MV1 (see FIG. 5 for plasmid map).

IPDC was amplified from Salmonella typhimurium with primers IPDC_accfwd and IPDC_sphrev, digested with Acc65I and SphI, and then was used to replace the KIVD fragment of plasmid pIBA8 (Zhang et al., ChemSusChem 4:1068-1070 (2011)) to form plasmid of p4MV2. Based on the crystal structure of KIVD active sites (FIG. 3a) and published data (Zhang et al., ChemSusChem 4:1068-1070 (2011)), on top of V461A, site-specific mutagenesis of F381L and F382L on KIVD were performed with oligos of kivd_F381Lfwd and kivd_F382Lfwd, and their corresponding reverser primers. M538A and M542L site-specific mutagenesis of KIVD were obtained using primers of kivd_accfwd with kivd_M538Arev and kivd_M542Lrev. The fragments of KIV mutagenesis of F381L, F382L, M538A and M542L were used to replace IPDC fragment in the plasmid p4MV2 to form p4MV3 (V461A/F381L) and p4MV4 (V461A/F382L), p4MV5 (V461A/M538A) and p4MV6 (V461A/M542L). According to the alignment results between KIVD and IPDC, V465A of IPDC was obtained using primers of IPDC_V465Afwd and IPDC_V465Arev. V540A and L544A site-specific mutagenesis of IPDC were obtained using primers of ipdc_accfwd with IPDC_V540Arev and IPDC_L544Arev. The fragments of IPDC mutagenesis of V465A, V540A and L544A were used to replace the wild-type IPDC fragment in the plasmid p4MV2 to form p4MV7 (V465A) and p4MV8 (V540A), and p4MV9 (L544A). To construct the plasmid of pZElac-LeuABCD-IPDC(L544A)-kdh_{ba}(p4MV 10) and pZElac-LeuA*BCD-IPDC(L544A)-kdh_{ba} (p4MV11), the fragments of wild-type LeuABCD and mutant LeuABCD were obtained from Acc65I digested plasmids of pZE_LeuABCDKA6 carrying wild-type LeuABCD and mutant LeuABCD, respectively, and then these two fragments were inserted into Acc65I restriction site of p4MV9, respectively. The fragment of wild-type LeuABCD was inserted into the Acc65I site of pZAlac_ilvD_alsS to create p3MB1.

TABLE 1
Oligonucleotides for cloning.

| | | SEQ |
|---|---|---|
| | | ID |
| Name | sequence | NO: |
| kivd_F381Lfwd | GTTGCTGAACAAGGGACATCACTGTTTGGC | 56 |
| | GCTTCATCAATTTTC | |
| kivd_F381Lrev | GAAAATTGATGAAGCGCCAAACAGTGATGT | 57 |
| | CCCTTGTTCAGCAAC | |
| kivd_F382Lfwd | GCTGAACAAGGGACATCATTCCTGGGCGCT | 58 |
| | TCATCAATTTTCTTA | |
| kivd_F382Lrev | TAAGAAAATTGATGAAGCGCCCAGGAATGA | 59 |
| | TGTCCCTTGTTCAGC | |
| kivd_M538Arev | GGGCCCGCATGCTTATGATTTATTTTGTTC | 60 |
| | AGCAAATAGTTTGCCTGCTTTTTTCAGTA | |
| kivd_F542Lrev | GGGCCCGCATGCTTATGATTTATTTTGTTC | 61 |
| | AGCCAGTAGTTTGCCCATTTTTTTCAGTA | |
| IPDC_accfwd | GGGCCC GGTACC ATGCAAAACCCCTATA | 62 |
| | CCGTGGCCGA | |
| IPDC_sphrev | GGGCCC GCATGC TTATCCCCCGTTGCGG | 63 |
| | GCTTCCAGCG | |
| IPDC_V465Afwd | GCTGCTCAACAATGACGGCTATACCGCTGA | 64 |
| | GCGCGCCATTCACGGCGCGGCCCAGCGGT | |
| IPDC_V465Arev | ACCGCTGGGCCGCGCCGTGAATGGCGCGCT | 65 |
| | CAGCGGTATAGCCGTCATTGTTGAGCAGC | |
| IPDC_V540Arev | GGGCCCGCATGCTTATCCCCCGTTGCGGGC | 66 |
| | TTCCAGCGCCCGGGTCGCGGTACGCAGTA | |
| IPDC_L544Arev | GGGCCC GCATGCTTATCCCCCGTTGCGGG | 67 |
| | CTTCCGCCGCCCGGGTCACGGTACGCAGTA | |
| padA_bamfwd | GACTAT GGATCC ATGACAGAGCCGCATG | 68 |
| | TAGCAGT | |
| padA_bamrev | GACTAT GGATCC TTAATACCGTACACAC | 69 |
| | ACCGACTTAGTT | |
| IPDC_bamfwd | GGGCCC GGATCC ATGCAAAACCCCTATA | 70 |
| | CCGTGGCCGA | |
| IPDC_bamrev | GGGCCC GGATCC TTATCCCCCGTTGCGG | 71 |
| | GCTTCCAGCG | |

### 2. Collection of Homoketoleucine Substrate

The strain carrying only one plasmid of p4MV1 was fermented in 125 mL flask for 48 hours. The fermentation product of homoketoleucine was collected the fraction system of Agilent 1260 Infinity HPLC. The pH of the homoketoleucine collection was adjusted to 2.0 and then ethyl acetate was added to extract the homoketoleucine. The top layer carrying the ethyl acetate and homoketoleucine was transferred into 1.5 mL tubes, and then these tubes were evaporated in the vacuum machine. The homoketoleucine was quantified for later use.

### 3. Protein Expression and Purification

The gene fragments of kdh_{ba} and the wild-type IPDC were PCR amplified using p4MV2 plasmid as template with primers of kdh_{ba—}barnfwd and kdh_{ba—}bamrev, IPDC_bamfwd and IPDC_bamrev, respectively. The fragment of IPDC L544A mutation was PCR amplified using p4MV9 plasmid as template with primers of IPDC_banifwd and IPDC_bamrev. After digestion with BamHI, the three gene fragments were inserted into expression plasmid pQE9 (Qiagen; Valencia, Calif.) to yield pQE9-kdh_{ba}, pQE9_IPDC and pQE9-IPDCL544A. These plasmids of pQE9-kdh_{ba}, pQE9_IPDC and pQE9-IPDCL544A were transformed into E. coli strain BL21. 2 mL overnight pre-cultured BL21 cells were inoculated in 200 ml 2XYT rich medium containing 50 mg/L ampicillin and 25 mg/L kanamycin. 0.1 mM IPTG was added into the medium to induce the recombinant proteins expression. Cell pellets were lysed by sonication in a buffer containing 250 mM NaCl, 2 mM DTT, 5 mM imidazole and 50 mM Tris pH 9.0, and then using Ni-NTA column chromatography to purify the enzymes from crude cell lysates by applying a stepwise gradient of imidazole (up to 250 mM). The Amicon Ultra centrifugal filters (Millipore; Billerica, MA) were used to obtain the fractions of highest purity by exchanged buffer. The concentrated protein solutions, in which kdh_{ba} was stored at buffer 1 (50 μM Tris buffer, pH 8.0, 1 mM MgSO4, and 20% glycerol), wild-type IPDC and L544A [PDC was kept at buffer 2 (50 μM Tris buffer, pH 8.0, 1 mM MgSO4, 0.2 mM ThDP, and 20% glycerol), were flash frozen at −80° C. for long term storage.

4. Enzyme Assay of kdh_{ba}, wild-type IPDC and IPDC of L544A

Substrates of isobutyraldehyde, isovaleraldehyde, isocaproaldehyde, ketovaline and ketoleucine were purchased from Fisher, and NAD⁺was from New England Biolabs (Ipswich, Mass.). Substrate of homoketoleucine was collected by HPLC fraction system. Protein concentration was determined by measuring UV absorbance at 280 nm. The reaction mixture contained 0.5 mM NAD⁺ and 0.2-4 mM aldehyde in assay buffer (50 mM NaH2PO4, pH 8.0, 1 mM DTT) with a total volume of 80 μL. The reactions were started by adding 2 μL kdh_{ba} (final enzyme concentration 25 nM), and the generation of NADH was monitored at 340 nm (extinction coefficient, 6.22 mM⁻¹ cm⁻¹). The decarboxylation activity of IPDC and IPDC L544A mutant were measured at 30° C. using a coupled enzymatic assay method. Excess kdh_{ba} was used to oxidize aldehyde into acid, and concomitantly, cofactor NAD⁺ was reduced to NADH. The assay mixture contained 0.5 mM NAD⁺, 0.1 μM kdh_{ba} and 0.2-4 mM 2-keto acids in assay buffer (50 mM NaH2PO4, pH 6.8, 1 mM MgSO4, 0.5 mM ThDP) with a total volume of 80 μL. The reactions were started by adding 2 μL IPDC or IPDC L544A mutation (final concentration 25 nM), and the generation of NADH was monitored at 340 nm. Kinetic parameters (K_{cat} and Kₘ) were determined by fitting initial velocity data to the Michaelis-Menten equation using Origin software.

### 5. Product Distribution in Shake Flask Fermentation

Table 2 shows the products produced by the various constructs. The host strain is AKO1. Certain alcohols were produced in addition to the indicated organic acids. Thus, the cells still exhibit some detectable level of alcohol dehydrogenase activity.

TABLE 2
Major products of isocaproate fermentation in shake flask.

| | Titer (g/L) | | | | | |
|---|---|---|---|---|---|---|
| | | 3-methy- | | 3-methyl- | 4-methyl- | |
| Strain | isobutyrate | butyrate | isobutanol | butanol | pentanol | isocaproate |
| WT KIVD + AldB | 0.74 ± 0.094 | 0.17 ± 0.006 | 0.32 ± 0.068 | 1.21 ± 0.074 | 1.07 ± 0.058 | 0.24 ± 0.006 |
| WT KIVD + AldH | 3.72 ± 0.591 | 2.51 ± 0.581 | 0.16 ± 0.042 | 0.46 ± 0.255 | 0.23 ± 0.071 | 1.30 ± 0.398 |
| WT KIVD + KDHba | 1.26 ± 0.074 | 1.56 ± 0.135 | 0.64 ± 0.102 | 0.87 ± 0.058 | 0.04 ± 0.012 | 2.69 ± 0.066 |
| WT KTVD + PadA | 1.78 ± 0.079 | 1.80 ± 0.119 | 0.06 ± 0.005 | 0.74 ± 0.025 | 0.61 ± 0.104 | 1.88 ± 0.089 |
| KIVD (V461A/ | 0.28 ± 0.061 | 0.02 ± 0 | 0.02 ± 0 | 0.12 ± 0 | 0.01 ± 0.005 | 1.58 ± 0.082 |
| F381L) + PadA | | | | | | |
| KTVD (V461A/ | 0.13 ± 0.01 | 0 ± 0 | 0.02 ± 0.005 | 0.02 ± 0.005 | 0.02 ± 0.008 | 0.66 ± 0.011 |
| F382L) + PadA | | | | | | |
| KIVD (V461A/ | 0.26 ± 0.067 | 0.04 ± 0.006 | 0.02 ± 0 | 0.11 ± 0.006 | 0.02 ± 0.005 | 2.76 ± 0.032 |
| M538A) + PadA | | | | | | |
| KIVD (V461A/ | 0.23 ± 0.014 | 0.05 ± 0 | 0.06 ± 0.01 | 0.19 ± 0.005 | 0.02 ± 0.01 | 3.30 ± 0.027 |
| F542L) + PadA | | | | | | |
| WT IPDC + KDHba | 0.77 ± 0.014 | 2.48 ± 0.029 | 0.71 ± 0.025 | 0.74 ± 0.036 | 0.02 ± 0.005 | 2.67 ± 0.070 |
| EPDC(V465A) + KDHba | 0.06 ± 0.006 | 0.19 ± 0.029 | 0.18 ± 0.021 | 0.39 ± 0.016 | 0.01 ± 0.005 | 4.14 ± 0.189 |
| IPDC(V540A) + KDHba | 0.22 ± 0.028 | 0.29 ± 0.029 | 0.14 ± 0.01 | 0.70 ± 0.037 | 0.09 ± 0.012 | 4.35 ± 0.056 |
| EPDC(L544A) + KDHba | 0.21 ± 0.058 | 0.20 ± 0.109 | 0.09 ± 0.039 | 0.55 ± 0.101 | 0.20 ± 0.095 | 5.02 ± 0.014 |
| medium copy mutant | 0.04 ± 0.029 | 0.30 ± 0.055 | 0.10 ± 0.007 | 0.41 ± 0.071 | 0.03 ± 0.016 | 5.53 ± 0.136 |
| leuABCD + high copy | | | | | | |
| WT leuABCD | | | | | | |
| medium copy mutant | 0.08 ± 0.010 | 0.05 ± 0.009 | 0.03 ± 0.010 | 0.11 ± 0.033 | 0.01 ± 0.001 | 3.70 ± 0.121 |
| leuABCD + high copy | | | | | | |
| mutant leuABCD | | | | | | |
| medium copy WT | 0.04 ± 0.009 | 1.45 ± 0.180 | 0.14 ± 0.045 | 0.70 ± 0.153 | 0.01 ± 0.003 | 4.61 ± 0.051 |
| leuABCD + high copy | | | | | | |
| mutant leuABCD | | | | | | |

### Example 2

E. coli strain AKO1 strain is BW25113 with ΔyqhD. E. coli strain AKO5 is BW25113 with Δpta, ΔpoxB, ΔadhE, ΔldhA, and ΔyqhD. Construction of plasmids is described in detail below.

For FIG. 8c, overnight culture was diluted 25-fold into 5 ml fresh medium in a 125-mL conical flask (0.5 g CaCO₃ was added to buffer the pH). Production was induced with 0.1 mM IPTG at 30° C. For bioreactor fermentation (FIG. 3a), production started once OD₆₀₀ reached 8.0 with the addition of 0.2 mM IPTG. The pH was controlled at 7.0 and the temperature was kept at 30° C. Dissolved oxygen (DO) level was maintained at 10% air saturation. Fermentation products were analyzed with an Agilent 1260 HPLC.

### 1. Plasmid Construction

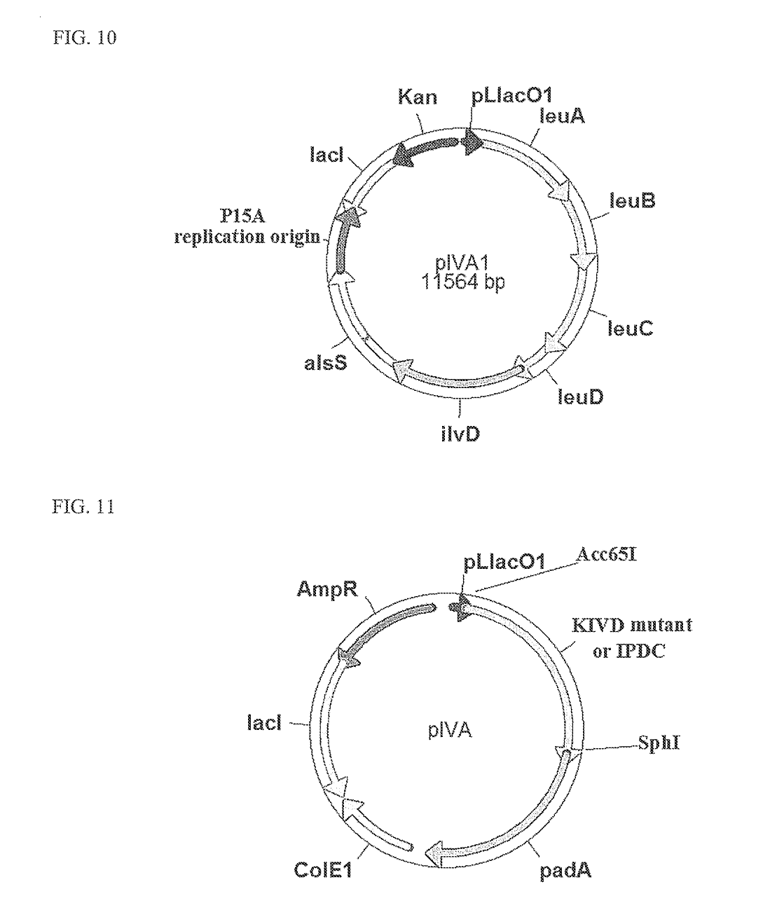

The 4702 by fragment of leuABCD operon was obtained from pZE_LeuABCDKA6 plasmid after Acc65I digestion, and inserted into pZAlac_iIvD_alsS (pIBA1) (Stephanopoulos et al., Metabolic engineering: principles and methodologies, (Academic Press, 1998)) to create pIVA1 (see FIG. 10 for plasmid map) (Chen et al., Appl. Catal. A: Gen. 169:207-214 (1998)).

Based on the crystal structure of KIVD active site (FIG. 12), on top of V461A mutant KIVD (Stephanopoulos et al., Metabolic engineering: principles and methodologies, (Academic Press, 1998)), site-specific mutagenesis of F381L and F382L were performed with oligo pairs kivd_F381Lfwd/kivd_F381Lrev and kivd_F382Lfwd/kivd_F382Lrev. While mutations M538A and F542L were obtained with primer pair kivd_accfwd and kivd_M538Arev or kivd_F542Lrev. The gene fragments of KIVD mutants were used to replace the wild-type KIVD fragment in pIBA7 plasmid (www.eastman.com/Literature_Center/M/M285.pdf) to form pIVA2 (V461A/F381L) and pIVA3 (V461A/F382L), pIVA4 (V461A/M538A) and pIVA5 (V461A/F542L). IPDC was amplified from the genomic DNA of Salmonella typhimurium with primers IPDC_accfwd and IPDC_sphrev, digested with Acc65I and SphI, and then inserted into the corresponding restriction site of pIVA2 to form plasmid pIVA6.

TABLE 3
Oligonucleotides for cloning.

| | | SEQ |
|---|---|---|
| | | ID |
| Name | sequence | NO: |
| kivd_F381Lfwd | GTTGCTGAACAAGGGACATCACTGTTTGGC | 72 |
| | GCTTCATCAATTTTC | |
| kivd F381Lrev | GAAAATTGATGAAGCGCCAAACAGTGATGT | 73 |
| | CCCTTGTTCAGCAAC | |
| kivd_F382Lfwd | GCTGAACAAGGGACATCATTCCTGGGCGCT | 74 |
| | TCATCAATTTTCTTA | |
| kivd_F382Lrev | TAAGAAAATTGATGAAGCGCCCAGGAATGA | 75 |
| | TGTCCCTTGTTCAGC | |
| kivd_M538Arev | GGGCCCGCATGCTTATGATTTATTTTGTTC | 76 |
| | AGCAAATAGTTTGCCTGCTTTTTTCAGTA | |
| kivd_F542Lrev | GGGCCCGCATGCTTATGATTTATTTTGTTC | 77 |
| | AGCCAGTAGTTTGCCCATTTTTTTCAGTA | |
| KIVD_accfwd | GACTAT GGTACC ATGTATACAGTAGGAG | 78 |
| | ATTACCTATTAG | |
| IPDC_accfwd | GGGCCC GGTACC ATGCAAAACCCCTATA | 79 |
| | CCGTGGCCGA | |
| IPDC_sphrev | GGGCCC GCATGC TTATCCCCCGTTGCGG | 80 |
| | GCTTCCAGCG | |
| IPDC_bamfwd | GGGCCC GGATCC ATGCAAAACCCCTATA | 81 |
| | CCGTGGCCGA | |
| IPDC_bamrev | GGGCCC GGATCC TTATCCCCCGTTGCGG | 82 |
| | GCTTCCAGCG | |

### 2. Protein Engineering Based on Crystal Structure of KIVD

To increase the production of isovalerate and decrease the formation of byproducts, the effect of enlarging the binding pocket of KIVD was investigated. According to the crystal structure (PDB: 2VBG), amino acid residues Phe-381, Phe-382, Val-461, Met 538 and Phe-542, in combination with the cofactor thiamine diphosphate (ThDP), delineate the active site of KIVD (de la Plaza et al., FEMS Microbiol. Lett. 238, 367-74 (2004)).

### 3. Protein Expression and Purification

IPDC gene fragment was PCR amplified with primers IPDC_bamfwd and IPDC_bamrev. After digestion with BamHI, the gene fragments were inserted into expression plasmid pQE9 (Qiagen; Valencia, Calif.) to yield pQE9-IPDC. The plasmid was transformed into BL-21 E. coli host harboring the pREP4 plasmid (Qiagen; Valencia, Calif.). Cells were inoculated from an overnight pre-culture at 1/100 dilution and grown in 200 ml 2XYT rich medium containing 50 mg/L ampicillin and 25 mg/L kanamycin. Protein expression was induced with 0.1 mM IPTG when OD₆₀₀ reached 0.6. After incubation at 30° C. overnight, cell pellets were lysed by sonication in a buffer containing 250 mM NaCl, 2 mM DTT, 5 mM imidazole and 50 mM Tris pH 9.0. The target protein was purified from crude cell lysates with Ni-NTA chromatography. Next, the protein solution was buffer-exchanged into storage buffer (50 μM Tris buffer, pH 8.0, 1 mM MgSO₄, 0.2 mM ThDP, and 20% glycerol) using Amicon Ultra centrifugal filters (Millipore). It was aliquoted (100 μl) into PCR tubes and flash frozen for long term storage at −80° C.

### 4. Product Distribution in Shake Flask Fermentation

Table 4 shows the products produced by the various constructs. The host strain is AKO1. Certain alcohols were produced in addition to the indicated organic acids. Thus, the cells still exhibit some detectable level of alcohol dehydrogenase activity.

TABLE 4
Major products of isovalerate fermentation in shake flask.

| | Titer (g/L) | | | |
|---|---|---|---|---|
| Strain | isobutyrate | isobutanol | 3-methyl-butanol | isovalerate |
| KIVD + AldB | 0.36 ± 0.007 | 0.24 ± 0.046 | 4.47 ± 0.046 | 0.76 ± 0.164 |
| KIVD + AldH | 0.37 ± 0.007 | 0.28 ± 0.039 | 0.37 ± 0.028 | 5.78 ± 0.530 |
| KIVD + KDHba | 0.42 ± 0.054 | 0.17 ± 0.017 | 2.10 ± 0.034 | 7.11 ± 0.013 |
| KIVD + PadA | 0.74 ± 0.075 | 0.11 ± 0.021 | 1.63 ± 0.082 | 7.53 ± 0.064 |
| KIVD (V461A/ | 0.10 ± 0.010 | 0.04 ± 0.028 | 1.63 ± 0.066 | 3.56 ± 0.069 |
| F381L) + PadA | | | | |
| KIVD (V461A/ | 0.16 ± 0.014 | 0.02 ± 0.000 | 0.66 ± 0.017 | 0.51 ± 0.016 |
| F382L) + PadA | | | | |
| KIVD (V461A/ | 0.12 ± 0.011 | 0.03 ± 0.010 | 1.45 ± 0.093 | 3.21 ± 0.204 |
| M538A) + PadA | | | | |
| KIVD (V461A/ | 0.07 ± 0.006 | 0.03 ± 0.005 | 2.03 ± 0.061 | 4.30 ± 0.326 |
| F542L) + PadA | | | | |
| IPDC + PadA | 0.51 ± 0.024 | 0.19 ± 0.005 | 1.19 ± 0.100 | 8.91 ± 0.278 |

### Reagents.

Chemicals were obtained from Sigma-Aldrich (St. Louis, Mo.) or Fisher Scientific (Waltham, Mass.). Restriction enzymes, DNA ligation kit and Phusion DNA polymerase were from New England Biolabs (Ipswich, Mass.). Rapid DNA ligation kit was from Roche (Madison, Wis.). Oligonucleotides were from Eurofms MWG Operon (Huntsville, Ala.).

### Strains and Plasmids.

Gene knockouts were introduced in BW25113 by P1 phage transduction using Keio collection. The knockout strain was transformed with plasmid pCP20 to remove the kanamycin resistance marker. The E. coil host was transformed with pIVA1 plasmid and another plasmid from pIBA4 to pIBA8 or pIVA2 to pfVA6 for isovalerate production. Construction of plasmids was carried out with standard molecular biology methods and is described in detail in the Supplementary information.

### Shake Flask Fermentation.

Overnight culture was diluted 25 fold into 5 ml M9 medium (plus 0.5% yeast extract and 4% glucose) in a 125-ml conical flask. Ampicillin (100 mg/L) and kanamycin (25 mg/L) were added to maintain the transformed plasmids. The culture medium was buffered with CaCO₃ powder. Isopropyl-b-D-thiogalactoside (IPTG) was added at a concentration of 0.1 mM to induce protein expression. The fermentation flasks were kept in a 30° C. shaker (250 rpm) and incubated for 48 hours.

### Bioreactor Fermentation.

Seeding medium has the following composition (g/L): glucose, 10; (NH₄)₂SO₄, 1.8; K₂HPO₄, 8.76; KH₂PO₄, 2.4; sodium citrate, 1.32; yeast extract, 15; ampicillin, 0.1; kanamycin, 0.05. Fermentation media for bioreactor cultures contained the following components (g/L): glucose, 30; (NH₄)₂SO₄, 3; K₂HPO₄, 14.6; KH₂PO₄, 4; sodium citrate, 2.2; yeast extract, 25; MgSO₄.7H₂O, 1.25; CaCl₂.2H₂O, 0.015, calcium pantothenate, 0.001; Thiamine, 0.01; ampicillin, 0.1; kanamycin, 0.05; and 1 mL/L of trace metal solution. Trace metal solution contained (g/L): NaCl, 5; ZnSO₄.7H₂O, 1; MnCl₂.4H₂O, 4; CuSO₄.5H₂O, 0.4; H₃BO₃, 0.575; Na2MoO4.2H₂O, 0.5; FeCl₃.6H₂O, 4.75; 6N H₂SO₄, 12.5 mL. The feeding solution contained (g/L): glucose, 600; (NH₄.)₂SO₄, 5; MgSO₄.7H₂O, 1.25; yeast extract, 5; CaCl₂.2H₂O, 0.015; calcium pantothenate, 0.001; Thiamine, 0.01; ampicillin, 0.1; kanamycin, 0.05, 0.2 mM IPTG; and 1 mL/L of trace elements.

Cultures of E. coli were performed in a 1.3 L Bioflo 115 fermenter (NBS; Edison, N.J.) using a working volume of 0.6 L. The fermenter was inoculated with 10% of overnight pre-culture with seeding medium and then the cells were grown at 37° C., 30% dissolved oxygen (DO), and pH 7.0. When OD₆₀₀ was 8.0, 0.2 mM IPTG was added and the temperature was decreased to 30° C. to start isovalerate production. The pH was controlled at 7.0 by automatic addition of 10 M NaOH solution. Air flow rate was maintained at 1 vvm in the whole process. Dissolved oxygen (DO) was maintained about 10% with respect to air saturation by raising stirring speed (from 300 to 800 rpm). The glucose level in the fermenter was kept about 10 g/L by inputting feeding medium continuously. Fermentation samples were collected to determinate concentrations of isovalerate, organic acids and glucose.

### Enzymatic Assay of PadA and IPDC.

Protein concentration was measured by UV absorbance at 280 nm. For PadA characterization, the assay buffer (50 mM NaH2PO4, pH 8.0, 1mM DTT) contained 0.5 mM NAD⁺ and 0.2-4 mM isopentanal. Enzymatic reactions were initiated by adding 25 nM PadA. The reactions were monitored by measuring the UV absorbance at 340 nm (extinction coefficient, 6.22 mM⁻¹ cm⁻¹) as a consequence of NADH generation.

The decarboxylation activity of IPDC was measured using a coupled enzymatic assay method. Excess PadA was added to the reaction mixture to quickly oxidize the aldehyde product into the corresponding acid, and concomitantly, cofactor NAD⁺was reduced to NADH. The assay mixture contained 0.5 mM NAD⁺, 0.1 μM PadA and 0.2-4 mM 2-keto acids in the reaction buffer (50 mM NaH2PO4, pH 6.8, 1 mM MgSO₄, 0.5 mM ThDP) with a total volume of 80 The reaction was started with the addition of 25 nM IPDC, and the progress was monitored by determining the generation of NADH (absorbance at 340 nm). Kinetic parameters (kcat and Km) were determined by fitting experimental velocity to Michaelis-Menten equation using Origin software.

### Example 3

### Cloning Procedure.

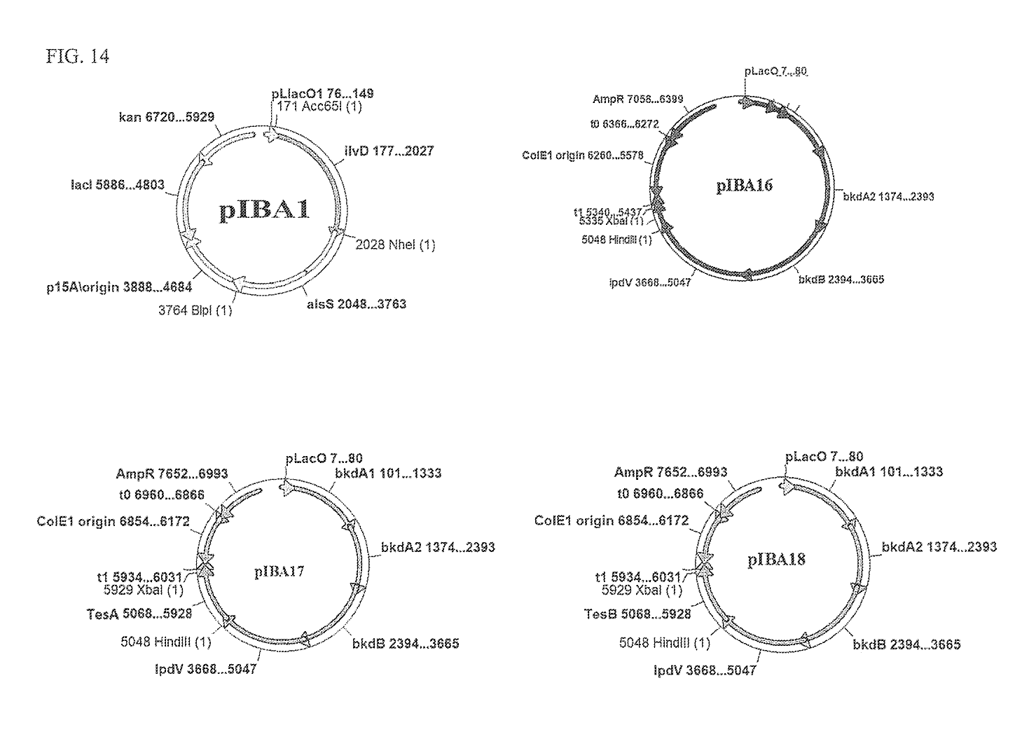

BKDH enzyme complex genes are amplified from Pseudomonas Putida KT2440 genomic DNA with primers bkdh_ecofwd (TGCATCGAATTCAGGAGAAATTAACTATGA ACGAGTACGCCCCCCTGCGTTTGC; SEQ ID NO:85) and bkdh_hindrev (TGCATCAAGC TTTCAGATATGCAAGGCGTGGCCCAG; SEQ ID NO:86). The PCR product was then digestion with EcoRI and Hindlll, and inserted into pZE12 to make pIBA16. The tesA gene was amplified from E. coli strain K12 genomic DNA using the primer pair TesAJffind111JF (GGGCCCAAGCTTAGGAGAAATTAACTATGATGAACTTCAACAATGTTTTCCG; SEQ ID NO:87) and TesA_Xbal_R (GGGCCCTCTAGATTATGAGTCATGATTTACTAAAGGCT; SEQ ID NO:88); tesB was amplified with primer pair TesB_Hindlll_F (GGGCCCAAGCTTAG GAGAAATTAACTATGATGAGTCAGGCGCTAAAAAATTTACT; SEQ ID NO:89) and TesB_Xbal_R (GGGCCCTCTAGATTAATTGTGATTACGCATCACCCCTT; SEQ ID NO:90). After PCR, the DNA fragments were purified and digested using the restriction enzymes Hindlll and Xbal. The digested fragments containing tesA are inserted into pIBA16 (FIG. 14) to make pIBA17 (FIG. 14); the digested fragments containing tesB are inserted into pIBA16 to make pIBA18 (FIG. 14).

The resulting plasmids were transformed into E. coli BW25113 for fermentation analysis.

### Fermentation Process.

Overnight cultures incubated in LB medium were diluted 25-fold into 5 mL M9 medium supplemented with 0.5% yeast extract and 4% glucose in 125-mL conical flasks. Antibiotics were added appropriately (ampicillin 100 mg/L and kanamycin 25 mg/L). 0.1 mM isopropyl-b-D-thiogalactoside (IPTG) was added to induce protein expression. The culture medium was buffered by addition of 0.5 g CaCO₃. Cultures were placed in a 30° C. shaker (250 rpm) and incubated for 48 hours.

Fermentation products were quantified by HPLC analysis with refractive index detection using an Agilent 1100 Capillary HPLC. Results are shown in Table 5.

TABLE 5
Production of isobutyrate with the new pathway

| Modification | Isovalerate (g/L) |
|---|---|
| alsSilvDLeuABCD | 0.56 ± .03 |
| alsSilvDLeuABCD + BKDH | 2.65 ± .03 |
| alsSilvDLeuABCD + BKDHTesA | 3.22 ± .69 |
| alsSilvDLeuABCD + BKDHTesB | 10.27 ± .33 |

### Example 4

### Cloning of Branched-Chain Keto-Acid Dehydrogenase Gene from Pseudomonas putida and Thioesterase II Gene from E. coli and Their Overexpression in Saccharomyces cerevisiae

The purpose of this Example is to describe the cloning of genes encoding P. putida branched-chain ketoacid dehydrogenase (BKDH) subunits, co-expressed with an E. coli gene encoding thioesterase II (TesB), under the control of a constitutively active promoter, and to describe the expression of such genes in an S. cerevisiae host strain.

For the cloning of BKDH subunits, genes are amplified using Pseudomonas putida genomic DNA and bkdh_ecofwd (TGCATCGAATTCAGGAGAAATTAACTATGAACGAGT ACGCCCCCCTGCGTTTGC; SEQ ID NO:85) and bkdh_hindrev (TGCATCAAGCTTTCAGA TATGCAAGGCGTGGCCCAG; SEQ ID NO:86). For the cloning of the E.coli tesB gene, genomic DNA of E. coli is amplified using primers TesB_Hindlll_F (GGGCCCAAGCTTAGGA GAAATTAACTATGATGAGTCAGGCGCTAAAAAATTTACT; SEQ ID NO:89) and TesB_Xbal_R (GGGCCCTCTAGATTAATTGTGATTACGCATCACCCCTT; SEQ ID NO:90) After PCR, the DNA fragments are purified and digested using the restriction enzymes Hindlll and Xbal. The digested fragments containing tesB are inserted into pIBA16 (FIG. 14) to make pIBAI8 (FIG. 14). The resulting plasmid (pIBA18) is utilized to transform into yeast strain Saccharomyces cerevisiae (W303a).

Saccharomyces cerevisiae (W303a) transformations are done using the lithium acetate method (Gietz, 2002, Methods in Enzymology, 350:87-96). One mL of an overnight yeast culture is diluted into 50 mL of fresh YPD medium and incubated in a 30° C. shaker for approximately six hours. The cells are collected, washed with 50 mL of sterile water, and washed again with 25 mL of sterile water. The cells are then resuspended using 1 mL of 100 mM lithium acetate and transferred to a new microcentrifuge tube. The cells are centrifuged for ten seconds in order to pellet them. The supernatant is then discarded and the cells are resuspended 4× in 100 mM lithium acetate. Fifteen microliters of cells are added to a DNA mix that consists of 72 μL 50% PEG, 10 μL 1M lithium acetate, 3 μL 10g/L denatured salmon sperm DNA, 2 μL of the desired plasmid DNA, and sterile water to a total volume of 100 μL. The samples are incubated at 30° C. for 30 minutes and heat shocked at 42° C. for 22 minutes. The cells are collected through centrifugation for ten seconds, resuspended in 100 μL SOS medium and plated onto appropriate SC selection plates without uracil, tryptophan, leucine, or histidine.

The complete disclosure of all patents, patent applications, and publications, and electronically available material (including, for instance, nucleotide sequence submissions in, e.g., GenBank and RefSeq, and amino acid sequence submissions in, e.g., SwissProt, MR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A recombinant microbial cell modified to exhibit increased biosynthesis of isocaproate compared to a wild-type control.

2. A recombinant microbial cell modified to exhibit increased biosynthesis of isovalerate compared to a wild-type control.

3. The recombinant microbial cell of claim 1 wherein the microbial cell is a fungal cell.

4-5. (canceled)

6. The recombinant microbial cell of claim 1 wherein the microbial cell is a bacterial cell.

7-19. (canceled)

20. The recombinant microbial cell of claim 1 wherein the microbial cell is photosynthetic.

21. The recombinant microbial cell of claim 1 wherein the microbial cell is cellulolytic.

22. The recombinant cell of claim 1 wherein the increased biosynthesis of isocaproate compared to a wild-type control comprises an increase in 2-isopropylmalate synthase activity compared to a wild-type control, an increase in ketoleucine elongation activity compared to a wild-type control, an increase in ketoacid decarboxylase activity compared to a wild-type control, an increase in ketoacid decarboxylase selectivity toward a predetermined substrate compared to a wild-type control, or an increase in aldehyde dehydrogenase activity compared to a wild-type control.

23-25. (canceled)

26. The recombinant cell of claim 2 wherein the increased biosynthesis of isovalerate compared to a wild-type control comprises increased leucine biosynthesis compared to a wild-type control, increased decarboxylase activity compared to a wild-type control, increased aldehyde dehydrogenase activity compared to a wild-type control, or increased branched-chain ketoacid dehydrogenase activity compared to a wild-type control.

27-28. (canceled)

29. A method comprising:
incubating the recombinant cell of claim 1 in medium that comprises a carbon source under conditions effective for the recombinant cell to produce isocaproate, wherein the carbon source comprises one or more of: glucose, pyruvate, ketovaline, ketoleucine, ketohomoleucine, CO₂, cellulose, xylose, sucrose, arabinose, or glycerol.

30. A method comprising:
incubating the recombinant cell of claim 2 in medium that comprises a carbon source under conditions effective for the recombinant cell to produce isovalerate, wherein the carbon source comprises one or more of: glucose, pyruvate, ketovaline, ketoleucine, isopentanal, CO₂, cellulose, xylose, sucrose, arabinose, or glycerol.

31. A method comprising:
introducing into a host cell a heterologous polynucleotide encoding at least one polypeptide that catalyzes conversion of a carbon source to isocaproate, wherein the at least one polypeptide is operably linked to a promoter so that the modified host cell catalyzes conversion of the carbon source to isocaproate.

32. The method of claim 31 wherein the carbon source comprises one or more of:
glucose, pyruvate, ketovaline, ketoleucine, ketohomoleucine, CO₂, cellulose, xylose, sucrose, arabinose, or glycerol.

33. A method comprising:
introducing into a host cell a heterologous polynucleotide encoding at least one polypeptide that catalyzes conversion of a carbon source to isovalerate, wherein the at least one polypeptide is operably linked to a promoter so that the modified host cell catalyzes conversion of the carbon source to isovalerate.

34. The method of claim 33 wherein the carbon source comprises one or more of:
glucose, pyruvate, ketovaline, ketoleucine, isopentanal, CO₂, cellulose, xylose, sucrose, arabinose, or glycerol.

35. The method of claim 31 wherein the host cell is a fungal cell.

36-37. (canceled)

38. The method of claim 31 wherein the host cell is a bacterial cell.

39-51. (canceled)

52. The method of claim 31 wherein the host cell is photosynthetic.

53. The method of claim 31 wherein the host cell is cellulolytic.

54. A method of harvesting an organic acid from a fermentation broth, the method comprising:
adjusting the pH of the fermentation broth to about 3.0;
adding an organic solvent to the fermentation broth, thereby producing an aqueous phase and a non-aqueous phase; and
extracting the organic acid from the aqueous phase.

55. The method of claim 54 wherein the organic solvent comprises hexane or oleyl alcohol.

56. The recombinant microbial cell of claim 2 wherein the microbial cell is a fungal cell.

57. The recombinant microbial cell of claim 2 wherein the microbial cell is a bacterial cell.

58. The recombinant microbial cell of claim 2 wherein the microbial cell is photosynthetic.

59. The recombinant microbial cell of claim 2 wherein the microbial cell is cellulolytic.

60. The method of claim 33 wherein the host cell is a fungal cell.

61. The method of claim 33 wherein the host cell is a bacterial cell.

62. The method of claim 33 wherein the host cell is photosynthetic.

63. The method of claim 33 wherein the host cell is cellulolytic.


SEQUENCE LISTING (93 sequences)

SEQ ID NO: 1 - protein, 550 aa, Salmonella bongori
MQTPYTVADYLLDRLAGCGIDHLFGVPGDYNLQFLDHVIDHPTLRWVGCANELNAAYAADGYARMSGAGALLTTFGVGELSAINGIAGSYAEYVPVLHIVGAPCSDAQQRGELMHHTLGDGDFRHFYRMSQAISAASAVLNEQNACYEIDRVLGEMLTAHRPCYILLPADVAKKPAIPPTETLMLPANKAQSSVETAFRYHARQCLMNSRRIALLADFLARRFGLRPLLQRWMVETPIAHATLLMGKGLFNEQHPNFVGTYSAGASSKEVRQAIEDADMVICVGTRFVDTLTAGFTQQLPAERTLEIQPYASRIGDSWFTLPMELAVSILRELCLECAFAPSPTRSSGQSIPVEKGALTQENFWQTLQQFIKPGDIILVDQGTAAFGAAALSLPDGAEVLVQPLWGSIGYSLPAAFGAQTACPDRRVILIIGDGAAQLTIQEMGSMLRDEQAPIILLLNNEGYTVERAIHGAAQRYNDIASWNWTQIPQALSAAQQAECWRVTQAIQLEEILARLARPQRLSLIEVMLPKADLPELLRTVTRALEMRNGG

SEQ ID NO: 2 - protein, 516 aa, Salmonella enterica
LDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQCLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 3 - protein, 550 aa, Salmonella enterica
MQTPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFHHFYRMSQAISAGSAILNEQNACFEIDRVLGEMVAARRPGYIMLPADVAKKTAIPPIEALTLPAHETQNGVETAFRYRARQCLMNSRRIALLADFLARRFGLRPLLQRWMAETSIAHATLLMGKGLFDEQHPNFVGTYSAGASSKAVRQAIEDADMVICVGTRFVDTLTAGFTQQLPAERTLEIQPYASRIGETWFNLPMAQAVSTLRELCLECAFAPPPTRPVCQPVQIEKGELTQENFWQTLQQYLKPGDIILVDQGTAAFGAAALSLPDGAEVVVQPLWGSIGYSLPAAFGAQTACPDRRVILIIGDGAAQLTIQEMGSMLRDGQAPIILLLNNDGYTVERAIHGAAQRYNDIASWNWTQIPQALNAAQQAECWRVTQAIQLAEVLERLARPQRLSFIEVMLPKADLPELLRTVTRALEARNGG

SEQ ID NO: 4 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALTLPVHEAQSGVETAFRYHARQCLMNSRRIALLADFLAGRFGLRPLLQRWMAETPIAHATLLMEKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNGGYTVERAIHGAAQRYNDIASWNWTQIPPALNAAQQVECWRVAQAIQLAEVLERLARPQRLSFIEVMLPKADLPELLRTVTRALEARNGG

SEQ ID NO: 5 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILDEQNACFEIDRVLGEMFAARRPGYIMLPADVAKKTAIPPTQALTLPVHEAQSGVETAFRYHARQCLMNSRRIALLADFLAGRFGLRPLLQRWMVETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLVRPQRLSFIEVMLPKADLPELLRTVTRALEARNGG

SEQ ID NO: 6 - protein, 549 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILYEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSTGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNG

SEQ ID NO: 7 - protein, 550 aa, Salmonella enterica
MQNPYTVADYLLDRLAGCGIGHLFGVPGDYNLQFLDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQCLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 8 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISVASAILDEQNACFEIDRVLGEMLVARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTVGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAKVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 9 - protein, 550 aa, Salmonella enterica
MQNPYTVADYLLDRLAGCGIGHLFGVPGDYNLQFLDHVIDHPTLRWVGCANELNAAYAADGYARMSGTGALLTTFGVGELSAINGIAGSYAEYVPVLHIVGAPCSAAQQRGELMHHTLGDGDFRHFYRMSQAISVASSILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 10 - protein, 550 aa, Salmonella enterica
MQNPYTVADYLLDRLAGCGIGHLFGVPGDYNLQFLDHVIDHPTLRWVGCANELNAAYAADGYARMSGTGALLTTFGVGELSAINGIAGSYAEYVPVLHIVGAPCSAAQQRGELMHHTLGDGDFRHFYRMSQAISVASSILDEQNACFEIDRVLGEMLAARRPGYIMLPADVAKKTAIPPTEALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILVIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 11 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYALRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKAELPELLRTVTRALEARNGG

SEQ ID NO: 12 - protein, 550 aa, Salmonella enterica
MQNPYTVADYLLDRLAGCGIGHLFGVPGDYNLQFLDHVIDHPTLRWVGCANELNAAYTADGYARMSGAGALLTTFGVGELSAINGIAGSYAEYVPVLHIVGAPCSAAQQRGELMHHTLGDGDFRHFYRMSQAISVASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQGIQLAEVLERLARPQRLSFIEVMLPKADLPELLRTVTRALEARNGG

SEQ ID NO: 13 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAKVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 14 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAARRYNDIASWNWTQIPPALNAAQQAECWRVTQAIQLAEVLERLARPQRLSFIEVMLPKADLPELLRTVTRALEARNGG

SEQ ID NO: 15 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGAIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 16 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMFAARRPGYIMLPADVAKKTAIPPTQALTLPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 17 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTARFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 18 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIVLVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 19 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPTERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 20 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVVQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 21 - protein, 550 aa, Salmonella enterica
MQNPYTVADYLLDRLAGCGIGHLFGVPGDYNLQFLDHVIDHPTLRWVGCANELNAAYAADGYARMSGAGALLTTFGVGELSAINGIAGSYAEYVPVLHIVGAPCSAAQQRGELMHHTLGDGDFRHFYRMSQAISAASAILDEQNACFEIDRVLGEMLAARRPGYIMLPADVAKKTAIPPTQALALPVHEAQSGVETAFRYHARQCLMNSRRIALLADFLAGRFGLRPLLQRWMAETPIAHATLLMGKGLFDEQHPNFVGTYSAGASSKEVRQAIEDADRVICVGTRFVDTLTAGFTQQLPAERTLEIQPYASRIGETWFNLPMAQAVSTLRELCLECAFAPPPTRSAGQPVRIDKGELTQESFWQTLQQYLKPGDIILVDQGTAAFGAAALSLPDGAEVVLQPLWGSIGYSLPAAFGAQTACPDRRVILIIGDGAAQLTIQEMGSMLRDGQAPVILLLNNDGYTVERAIHGAAQRYNDIASWNWTQIPPALNAAQQAECWRVTQAIQLAEVLERLARPQRLSFIEVMLPKADLPELLRTVTRALEARNGG

SEQ ID NO: 22 - protein, 480 aa, Variovorax paradoxus
MTATYTDTRLLIDNEWVDATGGKTLDVVNPATGKVIGKVAHASIADLDRALAAAQRGFDKWRNTPANERAAVMRRAAGLIRERAGDIAKLLTQEQGKPLAEAKGETLAAADIIEWFADEGRRVYGRIVPSRNLAAQQLVLKEPLGPVAAFTPWNFPINQIVRKLGAALATGCSFLVKAPEETPASPAALLQAFVDAGIPPGTVGLVFGNPAEISNYLIAHPIIRKVTFTGSTPVGKQLAALAGSHMKRVTMELGGHAPVIVAEDADVALAVKAAGAAKFRNAGQVCISPTRFLVHNSLREEFARTLVKYTEGLKLGDGLAEGTTIGPLANARRLTAMAYVLEDARKKGATVAAGGERVGDSGNFFAPTVLTDVPLDADVFNNEPFGPIAAIRGFDTLEEAIAEANRLPFGLAGYAFTKSIKSAHLLSQKLELGMLWINQPATPSPEMPFGGVKDSGYGSEGGPEALEAYLNTKAVSILGV

SEQ ID NO: 23 - protein, 480 aa, Variovorax paradoxus
MTATYTDTRLLIDNEWVDATGGKTLDVVNPATGKAIGKVAHASIADLDRALAAAQRGFEKWRNTPANERAAVMRRAAGLIRERAPEIAKLLTQEQGKPLAEAKGETLAAADIIEWFADEGRRVYGRIVPSRNLAAQQLVIKEPLGPVAAFTPWNFPINQIVRKLGAALATGCSFLVKAPEETPASPAALLQAFVDAGIPPGTVGLVFGNPAEISNYLISHPIIRKVTFTGSTPVGKQLAALAGSHMKRVTMELGGHAPVIVAEDADVALAVKAAGAAKFRNAGQVCISPTRFLVHNSLREEFARTLVKYTEGLKLGDGLAEGTTLGPLANARRLTAMAHVLDDARKKGATVAAGGERVGDTGNFFAPTVLTDVPLDADVFNNEPFGPIAAIRGFDTLEEAIAEANRLPFGLAGYAFTRSIKNAHLLSQKLELGMLWINQPAAPSPEMPFGGVKDSGYGSEGGPEALEAYLNTKAVSIMSV

SEQ ID NO: 24 - protein, 481 aa, Burkholderia sp. H160
MAISSYTDTRLLINGEWCDAVSGKTLDVINPATGQAIGKVAHAGIADLDRALDAAQRGFEAWRKVPAHERATIMRKAAALVRERAADIGRLMTQEQGKPFAEARVEVLAAADIIEWFADEGRRVYGRIVPSRNLAAHSQVLKEPIGPVAAFTPWNFPVNQVVRKLSASLACGCSFLVKAPEETPASPAALLQAFVEAGVPPGTVGLVFGDPAEISSYLIPHPVIRKVTFTGSTPVGKQLAALAGTHMKRATMELGGHAPVIVAEDADVALAVKAAGAAKFRNAGQVCISPTRFLVHNSIREEFAAALVKHAESLKLGDGLAEGTTLGPLANARRLSAMAKVVEDARKTGAKVATGGERVGSEGNFFAATVLTDVPLEADVFNNEPFGPVAAIRGFDTLDEAITEANRLPYGLAGYAYTKSFANVHQLSQRMEVGMLWINQPATPTPEMPFGGVKDSGYGSEGGPEAMEAYLVTKAVTIMAV

SEQ ID NO: 25 - protein, 481 aa, Burkholderia rhizoxinica
MVTSSYTDTRLLIDGQWCDAASGKTLDVVNPATGQVIGRVAHAGIADLDRALAAAQRGFDTWRKVPVHERAATMRKAATLVRERAEGIARLMTQEQGKPFAEARIEVLSAADIIEWFADEGRRVYGRIVPSRNLAVQQSVLKEPIGPVAAFTPWNFPVNQVVRKLSAALACGCSFLVKAPEETPASPAGLLQAFVDAGVPAGTIGLVFGDPAAISSYLIAHPVIRKVTFTGSTPVGKQLAALAGAHMKRATMELGGHAPVIVAEDADIALAIKAAGGAKFRNAGQVCISPTRFLVHNSIREAFEAALVKHAQGLKLGDGLAQGTTLGPLANARRLTAMTRIVENARATGATVATGGERVGSAGNFFAPTVLTNVPRDADVFNQEPFGPVAAVRGFDRLEDAIAEANRLPYGLAGYAFTRSVRNVHLLSHQLEVGMLWINQPATPWPEMPFGGVKDSGYGSEGGPEAMEAYLVTKAVSVAAV

SEQ ID NO: 26 - protein, 481 aa, Burkholderia sp. CCGE1002
MAISSYTDTRLLINGEWCDAASGKTLDVINPATGQAIGKVAHAGIPDLDRALEAAQRGFEAWRKVPANERATIMRKAAALVRERASDIGRLMTQEQGKPFAEARVEVLAAADIIEWFADEGRRVYGRIVPSRNLAAQSQVLKEPIGPVAAFTPWNFPVNQVVRKLSASLACGCSFLVKAPEETPASPAALLQAFVEAGVPPGTVGLVFGDPAEISSYLIPHPVIRKVTFTGSTPVGKQLAALAGSHMKRATMELGGHAPVIVAEDADVALAVKAAGAAKFRNAGQVCISPTRFLVHNSIREEFAAALVKHAESLKLGDGLAEGTTLGPLANARRLSAMARVVDDARKTGAKVATGGERVGTEGNFFAATVLTDVPLEADVFNNEPFGPVAAIRGFDKLEEAIAEANRLPYGLAGYAYTKSFANVHLLSQRMEVGMLWINQPATPTPEMPFGGVKDSGYGSEGGPEAMEAYLVTKAVTVMSV

SEQ ID NO: 27 - protein, 481 aa, Burkholderia xenovorans
MAIPSYTDTRLLINGEWCDAASGKTLDVINPATGQAIGKVAHAGIADLDRALAAAQRGFEAWRKVPANERATTMRRAAALVRERASDIGRLMTQEQGKPFAEARIEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISGYLIPHPVIRKVTFTGSTPVGKQLAALAGAHMKRATMELGGHAPVIVAEDADVALAVKAAGGAKFRNAGQVCISPTRFLVHNSIREEFAAALVKHAEGLKLGDGLAEGTTLGPLANARRLSAMSKVLDDARKTGAKVETGGERVGSEGNFFAPTVLTNVSLEADVFNNEPFGPIAAIRGFDKLEEAIAEANRLPYGLAGYAFTKSFSNVHLLSQQVEVGMLWINQPATPSPEMPFGGVKDSGYGSEGGPEAMEGYLVTKAVSVMAV

SEQ ID NO: 28 - protein, 481 aa, Burkholderia sp. Ch1-1
MAISSYTDTRLLINGEWCDAASGKTLDVVNPATGQAIGKVAHAGIADLDRALAAAQRGFEAWRKVPANERATTMRRAAALVRERASDIGRLMTQEQGKPFAEARVEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISSYLIPHPVIRKVTFTGSTPVGKQLAALAGAHMKRATMELGGHAPVIVAEDADVALAVKAAGGAKFRNAGQVCISPTRFLVHNSIREEFAAALVKHAEGLKLGDGLAEGTTLGPLANARRLTAMSKVLDDARKTGAKVETGGERVGSEGNFFAPTVLTNVSLEADVFNNEPFGPIAAIRGFDKLEEAIAEANRLPYGLAGYAFTKSFSNVHLLSQQLEVGMLWINQPATPSPEMPFGGVKDSGYGSEGGPEAMEGYLVTKAVSVMAV

SEQ ID NO: 29 - protein, 481 aa, Burkholderia phytofirmans
MATSSYTDTRLLINGEWCDAASGKTLDVINPATGKAIGKVAHAGIADLDRALAAAQRGFEAWRKVPANERATTMRKAAALVRERASDIGRLMTLEQGKPFAEARIEVLAAADIIEWFADEGRRVYGRIVPSRNLAAQQLVLKEPIGPVAAFTPWNFPVNQVVRKLSAALACGCSFLVKAPEETPASPAALLQAFVEAGVPAGTVGLVFGDPAEISSYLIPHPVIRKVTFTGSTPVGKQLAALAGSHMKRATMELGGHAPVIVAEDADVALAVKAAGGAKFRNAGQVCISPTRFLVHNSIREEFAAALVKHAEGLKLGDGLAEGTTLGPLANARRLTAMSKVLDDARKTGAKVETGGERVGSEGNFFAPTVLTNVSLESDVFNNEPFGPIAAIRGFDKLEEAIAEANRLPFGLAGYAFTKSFTNVHLLSQQLEVGMLWINQPATPSPEMPFGGVKDSGYGSEGGPEAMEGYLVTKAVSVMSV

SEQ ID NO: 30 - protein, 481 aa, Burkholderia sp. CCGE1003
MAISSYTDTRLLINGEWCDAASGKTIDVLNPATGQVIGTVAHAGIADLDRALEAAQRGFEAWRKVPAHERAAVMRKAAALVRERASDIGRLMTQEQGKPFAEAKIEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISGYLIPHPVIRKVTFTGSTPVGKQLAALAGAHMKRATMELGGHAPVIVADDADVALAVKAAGGAKFRNAGQVCISPTRFLVHNSIREEFAAALVKHAESLKLGDGLAEGTTLGPLANARRLTAMSKVLEDARKTGAKVETGGERVGSEGNFFAPTVLTNVSLEADVFNNEPFGPIAAIRGFDKLEEAIAEANRLPFGLAGYAFTKSFSNVHLLSQQLEVGMLWINQPATPTPEMPFGGVKDSGYGSEGGPEAMEAYLVTKAVTVMSS

SEQ ID NO: 31 - protein, 481 aa, Burkholderia graminis
MAISSYTDTRLLINGEWCDAASGKTLDVLNPATGQVIGKVAHAGIADLDRALEAAQRGFEAWRKVPAHERAAVMRKAAALVRERASDIGRLMTQEQGKPFAEAKVEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISNYLIPHPVIRKVTFTGSTPVGKQLASLAGAHMKRATMELGGHAPVIVAEDADVALAVKAAGGAKFRNAGQVCISPTRFLVHNSIREEFAAALVKHAEGLKLGDGLADGTTLGPLANARRLTAMSKVLDDARRTGAKIETGGERVGTEGNFFAPTVLTNVSLEADVFNNEPFGPIAAIRGFDKLEEAIAEANRLPFGLAGYAFTKSFANVHLLSQQLEVGMLWINQPATPTPEMPFGGVKDSGYGSEGGPEAMEAYLVTKAVTVMSS

SEQ ID NO: 32 - protein, 482 aa, Burkholderia phymatum
MVTSSSYTDTRLLINNEWCDAASGKTLDVVNPATGKPIGKVAHAGKADLDRALEAAQKGFEAWRKVPANERATTMRKAAGFVRERADHIARLMTQEQGKPFAEARIEVLSAADIIEWFADEGRRVYGRVVPSRNLNAQSLVIKEPIGPVAAFTPWNFPVNQVVRKLSAALASGCSFLVKAPEETPASPAQLLQAFVDAGVPAGTVGLVFGDPAEISSYLIPHPVIRKVTFTGSTPVGKQLAALAGSHMKRATMELGGHAPVIVAEDADVALAVKAAGGAKFRNAGQVCISPTRFLVHNSIREAFAAALVKHAEGLKVGDGLAEGTQLGPLANARRLTAMASIIDNARSTGATVATGGERIGSEGNFFAPTVLTDVPLEADVFNNEPFGPIAAIRGFDNIEDAIAEANRLPFGLAGYAFTKSFRNVHLLSQNLEVGMLWINQPATPTPEMPFGGVKDSGYGSEGGPEAMEAYLVTKAVTVMAV

SEQ ID NO: 33 - protein, 480 aa, Burkholderia sp. CCGE1001
MAISSYTDTRLLINGEWCDAASGKTIDVLNPATGQVIGKVAHAGIADLDRALEAAQRGFEAWRKVPAHERAAVMRKAAALVRERASDIGRLMTQEQGKPFAEAKIEVLAAADIIEWFADEGRRLYGRVVPSRNLAAQQLVLKEPIGPVAAFTPWNFPVNQIVRKLSAALASGCSFLVKAPEETPASPAGLLQAFVEAGVPAGTVGLVFGDPAEISSYLIPHPVIRKVTFTGSTPVGKQLAALAGAHMKRATMELGGHAPVIVAEDADVALAVKAAGGAKFRNAGQVCISPTRFLVHNSIREEFAAALVKHAESLKLGDGLAEGTTLGPLANARRLTAMSKVLDDARKTGAKIETGGERVGSEGNFFAPTVLTNVSLEADVFNNEPFGPIAAIRGFDKLEEAIAEANRLPFGLAGYAFTKSFSNVHLLSQQLEVGMLWINQPATPTPEMPFGGVKDSGYGSEGGPEAMEAYLVTKAVTVMS

SEQ ID NO: 34 - protein, 481 aa, Burkholderia gladioli
MTNTTYTDTQLLINGEWCDAESGKTIDVLNPATGKVIGKVAHAGIADLDRALEAAQRGFETWRKVTAYDRAALMRKAAALVRERADTIAQLMTQEQGKPLVEAKIEVLSAADIIEWFADEGRRVYGRIVPPRNLAVQQTVVKEPVGPVAAFTPWNFPVNQVVRKLSAALATGCSFLVKAPEETPASPAQLLRAFVDAGVPAGVVGLVYGDPAEISNYLIPHPVIRKVTFTGSTPVGKQLAALAGQHMKRATMELGGHAPVIVAEDADLDLAVKAAGGAKFRNAGQVCISPTRFLVHNSVREDFAKALVKHAEGLKVGDGLALGTNLGPLANSRRLGAMEKVVADARKTGATVATGGERIGSEGNFFAPTVLTDVPLEADVFNNEPFGPIAAIRGFDSLEDAITEANRLPYGLAGYAFTRAFKNVHLLTQRLEVGMLWINQPATPWPEMPFGGVKDSGYGSEGGPEALEPYLVTKSVTVTAA

SEQ ID NO: 35 - protein, 481 aa, Burkholderia glumae
MTNTNYTDTQLLINGEWCDAASGKTLDVVNPATGQVIGKVAHAGIADLDRALDAAQRGFETWRKVSAYERSALMRKAAALVRERANSIAQLMTLEQGKPLAEARIEVLSAADIIEWFADEGRRVYGRIVPPRNLAVQQTVVKEPVGPVAAFTPWNFPVNQVVRKLSAALATGCSFLVKAPEETPASPAQLLRAFVDAGVPAGVVGLVYGDPAEISNYLIPHPVIRKITFTGSTPVGKQLAALAGQHMKRATMELGGHAPVIVAEDADLELAVKAAGGAKFRNAGQVCISPTRFLVHNSVREAFVKALVKHAEGLKVGDGLEAGTSLGPLANPRRLTAMEKVVADARKAGATVATGGERIGSAGNFFAPTVLADVPLDADVFNNEPFGPVAAVRGFDSLDDAITEANRLPYGLAGYAFTRSFKNVHLLTQRVEVGMLWINQPATPWPEMPFGGVKDSGYGSEGGPEALEPYLVTKSVTVAAV

SEQ ID NO: 36 - protein, 453 aa, Burkholderia cenocepacia
MNPATGKPIGKVAHAGIADLDRALAAAQRGFEAWRKVPAHERAATMRKAAALVRERADAIAQLMTQEQGKPLTEARVEVLSAADIIEWFADEGRRVYGRIVPPRNLNAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 37 - protein, 481 aa, Burkholderia ubonensis
MAHVTYTDTQLLINGEWTDAASGKTIDVVNPATGKAIGKVAHAGIADLDRALAAAQRGFEQWRRVPAHERAATMRKAAALVRERADGIAQLMTQEQGKPLVEARLEVLAAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISAYLIPHPVIRKVTFTGSTPVGKHLAALAGQHMKRATMELGGHAPVIVAEDADVALAVKAAGGAKFRNAGQVCISPTRFLVHNSIRDEFTRALVKHAQGLKVGNGLDEGTTLGALANPRRIAAMTSVVENARAVGARVETGGERIGTEGNFFAPTVLADVPLEADVFNNEPFGPVAAIRGFDSLDDAISEANRLPYGLAGYAFTRSFANVHLLTQRLEVGMLWINQPATPWPEMPFGGVKDSGYGSEGGPEALEPYLVTKSVTVMAV

SEQ ID NO: 38 - protein, 481 aa, Burkholderia sp. 383
MANVTYTDTQLLIDGEWVDAASGKTIDVVNPATGKPIGKVAHAGIADLDRALAAAQRGFDAWRKVPAHERAATMRKAAALVRERADAIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAAMAGLHMKRATMELGGHAPVIVAEDADVALAVKAAGGAKFRNAGQVCISPTRFLVHNSIRDEFTRALVKHAEGLKVGNGLEEGTALGALANPRRLTAMASVVDNARKVGARIETGGERIGTEGNFFAPTVIADVPLEADVFNNEPFGPVAAIRGFDKLDDAIAEANRLPFGLAGYAFTRSFANVHLLTQRLEVGMLWINQPATPWPEMPFGGVKDSGYGSEGGPEALEPYLVTKSVTVMAV

SEQ ID NO: 39 - protein, 483 aa, Burkholderia dolosa
MWMANVTYTDTQLLIDGEWVDAASGKTIDVVNPATGKAIGKVAHAGIADLDRALAAAQRGFEAWRKVPAHERAATMRKAAALVRERADTIAQLMTQEQGKPLAESRIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVFGDPAEISAYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGARIETGGERIGSEGNFFAPTVIADVPLEADVFNNEPFGPVAAIRGFDKLDDAIAEANRLPYGLAGYAFTRSFANVHLLTQRLEVGMLWINQPATPWPEMPFGGVKDSGYGSEGGPEALEPYLVTKSVTVMAV

SEQ ID NO: 40 - protein, 481 aa, Azospirillum brasilense
MANVTYTDTQLLIDGEWVDAASGKTIDVVNPATGKPIGRVAHAGIADLDRALAAAQSGFEAWRKVPAHERAATMRKAAALVRERADAIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLASLAGLHMKRATMELGGHAPVIVAEDADVALAVKAAGGAKFRNAGQVCISPTRFLVHNSIRDEFTRALVKHAEGLKVGNGLEEGTTLGALANPRRLTAMASVIDNARKVGASIETGGERIGSEGNFFAPTVIANVPLDADVFNNEPFGPVAAIRGFDKLEEAIAEANRLPFGLAGYAFTRSFANVHLLTQRLEVGMLWINQPATPWPEMPFGGVKDSGYGSEGGPEALEPYLVTKSVTVMAV

SEQ ID NO: 41 - protein, 481 aa, Burkholderia multivorans
MANVTYTDTQLLIDGEWVDAASGKTIDVVNPATGRVIGKVAHAGIADLDRALAAAQRGFEAWRKVPAHERAATMRKAAALVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQNMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 42 - protein, 481 aa, Burkholderia cenocepacia
MANVTYTDTQLLIDGEWVDAASGKTIDVVNPATGKPIGKVAHASIADLDRALAAAQRGFEAWRKVPAHERAATMRKAAALVRERADTIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 43 - protein, 481 aa, Burkholderia multivorans
MANVTYTDTQLLIDGEWVDAASGKTIDVVNPATGKVIGKVAHAGIADLDRALAAAQRGFEAWRKVPAHERAATMRKAAARVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 44 - protein, 481 aa, Burkholderia sp. TJI49
MANVTYTDTQLLIDGEWVDAASGKTIDVMNPATGKVIGKVAHAGIADLDRALAAAQRGFEAWRKVPAHERAATMRKAAALVRERADAIAQLMTQEQGKPLAEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 45 - protein, 481 aa, Burkholderia cenocepacia
MANVTYTDTQLLIDGEWVDAASGKTIDVVNPATGKPIGKVAHAGIADLDRALAAAQRGFEAWRKVPAHERAATMRKAAALVRERADTIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEEAIAEANRLPFGLAGYAFTRSFANVHLLTQRLEVGMLWINQPATPWPEMPFGGVKDSGYGSEGGPEALEPYLVTKSVTVMAV

SEQ ID NO: 46 - protein, 481 aa, Burkholderia multivorans
MANVTYTDTQLLIDGEWVDAASGKTIDVVNPATGKVIGKVAHAGIADLDRALAAAQRGFEAWRKVPAHERAATMRKAAALVRERADTIAQLMTQEQGKPLTEARIEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYVIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVENARKVGASVETGGERIGSEGNFFAPTVLANVPLEADVFNNEPFGPVAAIRGFDKLEDAIAEANRLPYGLAGYAFTRSFANVHLLTQRLEVGMLWINQPATPWPEMPFGGVKDSGYGSEGGPEALEPYLVTKSVTVMAV

SEQ ID NO: 47 - protein, 481 aa, Burkholderia vietnamiensis
MANVTYTDTQLLIDGEWVDAASGKTIDVVNPATGKAIGKVAHAGIADLDRALAAAQRGFEAWRKVPAHERAATMRKAAALVRERADAIAQLMTQEQGKPLTEARIEVLSAADIIEWFADEGRRVYGRIVPPRNLGAQQMVVKEPVGPVAAFTPWNFPVNQVVRKLCAALATGCSFLVKAPEETPASPAALLRAFVDAGVPAGVVGLVYGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVAHAQGLKIGNGLDEGTTLGALANPRRLTAMASVVENARKVGASIETGGERIGSEGNFFAPTVIANVPLEADVFNNEPFGPVAAIRGFDKLEDAISEANRLPFGLAGYAFTRSFANVHLLTQRLEVGMLWINQPATPWPEMPFGGVKDSGYGSEGGPEALEPYLVTKSVTVMAV

SEQ ID NO: 48 - protein, 481 aa, Burkholderia cenocepacia
MANVTYTDTQLLIDGEWVDAASGKTIDVVNPATGKPIGKVAHAGIADLDRALAAVQRGFEAWRKVPAHERAATMRKAAALVRERADTIAQLMTQEQGKPLTEARVEVLSAADIIEWFADEGRRVYGRIVPPRNFNAQQTVVKEPVGPVAAFTPWNFPVNQVVRKLSAALATGCSFLVKAPEETPASPAALLRAFVDAGVPAGVIGLVFGDPAEISSYLIPHPVIRKVTFTGSTPVGKQLAALAGQHMKRATMELGGHAPVIVAEDADVALAVKAAGGAKFRNAGQVCISPTRFLVHNSIRDEFTRALVKHAEGLKVGNGLEEGTTLGALANPRRLTAMASVVDNARKVGASIETGGERIGAEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 49 - protein, 481 aa, Burkholderia ambifaria
MANVTYTDTQLLIDGEWVDAASGKTIDVVNPATGKAIGKVAHAGIADLDRALAAAQRGFEAWRKVPANERAATMRKAAALVRERADTIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMVSVVDNARKVGARIETGGERIGSEGNFFAPTVIANVPLEADVFNNEPFGPVAAIRGFDKLDDAIAEANRLPFGLAGYAFTRSFANVHLLTQRLEVGMLWINQPATPWPEMPFGGVKDSGYGSEGGPEALEPYLVTKSVTVMAV

SEQ ID NO: 50 - protein, 481 aa, Burkholderia ambifaria
MANVTYTDTQLLIDGEWVDAASGKTIDVVNPATGKAIGKVAHAGIADLDRALVAAQRGFEAWRKVPANERAATMRKAAALVRERADTIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVDNARKVGASIETGGERIGSEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 51 - protein, 481 aa, Burkholderia ambifaria
MANVTYTDTQLLIDGEWVDAASGRTIDVVNPATGKAIGKVAHAGIADLDRALAAAQRGFEAWRKVPANERAATMRKAAALVRERADAIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGDPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVENARKVGASIETGGERIGSEGNFFAPTVIANVPLEADVFNNEPFGPVAAIRGFDKLEDAIAEANRLPFGLAGYAFTRSFANVHLLTQRLEVGMLWINQPATPWPEMPFGGVKDSGYGSEGGPEALEPYLVTKSVTVMAV

SEQ ID NO: 52 - protein, 481 aa, Burkholderia ambifaria
MANVTYTDTQLLIDGEWVDAASGKTIDVVNPATGKAIGKVAHAGIADLDRALAAAQRGFEAWRKVPANERAATMRKAAALVRERADAIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGEPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVENARKVGASIETGGERIGSEGNFFAPTVIANVPLEADVFNNEPFGPVAAIRGFDKLEDAIAEANRLPFGLAGYAFTRSFANVHLLSQRLEVGMLWINQPATPWPEMPFGGVKDSGYGSEGGPEALEPYLVTKSVTVMAV

SEQ ID NO: 53 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 54 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPYHSNAKGDLAQILQLTRYFAGSADKFDKGATIPLTFNKFAYTLKVPFGVVAQIVPWNYPLAMACWKLQGALAAGNTVIIKPAENTSLSLLYFATLIKKAGFPPGVVNIVPGYGSLVGQALASHMDIDKISFTGSTKVGGFVLEASGQSNLKDVTLECGGKSPALVFEDADLDKAIDWIAAGIFYNSGQNCTANSRVYVQSSIYDKFVEKFKETAKKEWDVAGKFDPFDEKCIVGPVISSTQYDRIKSYIERGKREEKLDMFQTSEFPIGGAKGYFIPPTIFTDVPQTSKLLQDEIFGPVVVVSKFTNYDDALKLANDTCYGLASAVFTKDVKKAHMFARDIKAGTVWINSSNDEDVTVPFGGFKMSGIGRELGQSGVDTYLQTKAVHINLSLDN

SEQ ID NO: 55 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPFHSNAKQDLAQIIELTRYYAGAVDKFNMGETIPLTFNKFAYTLKVPFGVVAQIVPWNYPLAMACRKMQGALAAGNTVIIKPAENTSLSLLYFATLIKKAGFPPGVVNVIPGYGSVVGKALGTHMDIDKISFTGSTKVGGSVLEASGQSNLKDITLECGGKSPALVFEDADLDKAIEWVANGIFFNSGQICTANSRVYVQSSIYDKFVEKFKETAKKEWDVAGKFDPFDEKCIVGPVISSTQYDRIKSYIERGKKEEKLDMFQTSEFPIGGAKGYFIPPTIFTDVPETSKLLRDEIFGPVVVVSKFTNYDDALKLANDTCYGLASAVFTKDVKKAHMFARDIKAGTVWINQTNQEEAKVPFGGFKMSGIGRESGDTGVDNYLQIKSVHVDLSLDK

SEQ ID NO: 56 - DNA, 45 bp, artificial
  synthetic oligonucleotide primer
gttgctgaacaagggacatcactgtttggcgcttcatcaattttc

SEQ ID NO: 57 - DNA, 45 bp, artificial
  synthetic oligonucleotide primer
gaaaattgatgaagcgccaaacagtgatgtcccttgttcagcaac

SEQ ID NO: 58 - DNA, 45 bp, artificial
  synthetic oligonucleotide primer
gctgaacaagggacatcattcctgggcgcttcatcaattttctta

SEQ ID NO: 59 - DNA, 45 bp, artificial
  synthetic oligonucleotide primer
taagaaaattgatgaagcgcccaggaatgatgtcccttgttcagc

SEQ ID NO: 60 - DNA, 59 bp, artificial
  synthetic oligonucleotide primer
gggcccgcatgcttatgatttattttgttcagcaaatagtttgcctgcttttttcagta

SEQ ID NO: 61 - DNA, 59 bp, artificial
  synthetic oligonucleotide primer
gggcccgcatgcttatgatttattttgttcagccagtagtttgcccatttttttcagta

SEQ ID NO: 62 - DNA, 38 bp, artificial
  synthetic oligonucleotide primer
gggcccggtaccatgcaaaacccctataccgtggccga

SEQ ID NO: 63 - DNA, 38 bp, artificial
  synthetic oligonucleotide primer
gggcccgcatgcttatcccccgttgcgggcttccagcg

SEQ ID NO: 64 - DNA, 59 bp, artificial
  synthetic oligonucleotide primer
gctgctcaacaatgacggctataccgctgagcgcgccattcacggcgcggcccagcggt

SEQ ID NO: 65 - DNA, 59 bp, artificial
  synthetic oligonucleotide primer
accgctgggccgcgccgtgaatggcgcgctcagcggtatagccgtcattgttgagcagc

SEQ ID NO: 66 - DNA, 59 bp, artificial
  synthetic oligonucleotide primer
gggcccgcatgcttatcccccgttgcgggcttccagcgcccgggtcgcggtacgcagta

SEQ ID NO: 67 - DNA, 59 bp, artificial
  synthetic oligonucleotide primer
gggcccgcatgcttatcccccgttgcgggcttccgccgcccgggtcacggtacgcagta

SEQ ID NO: 68 - DNA, 35 bp, artificial
  synthetic oligonucleotide primer
gactatggatccatgacagagccgcatgtagcagt

SEQ ID NO: 69 - DNA, 40 bp, artificial
  synthetic oligonucleotide primer
gactatggatccttaataccgtacacacaccgacttagtt

SEQ ID NO: 70 - DNA, 38 bp, artificial
  synthetic oligonucleotide primer
gggcccggatccatgcaaaacccctataccgtggccga

SEQ ID NO: 71 - DNA, 38 bp, artificial
  synthetic oligonucleotide primer
gggcccggatccttatcccccgttgcgggcttccagcg

SEQ ID NO: 72 - DNA, 45 bp, artificial; identical to SEQ ID NO: 56
  synthetic oligonucleotide primer

SEQ ID NO: 73 - DNA, 45 bp, artificial; identical to SEQ ID NO: 57
  synthetic oligonucleotide primer

SEQ ID NO: 74 - DNA, 45 bp, artificial; identical to SEQ ID NO: 58
  synthetic oligonucleotide primer

SEQ ID NO: 75 - DNA, 45 bp, artificial; identical to SEQ ID NO: 59
  synthetic oligonucleotide primer

SEQ ID NO: 76 - DNA, 59 bp, artificial; identical to SEQ ID NO: 60
  synthetic oligonucleotide primer

SEQ ID NO: 77 - DNA, 59 bp, artificial; identical to SEQ ID NO: 61
  synthetic oligonucleotide primer

SEQ ID NO: 78 - DNA, 40 bp, artificial
  synthetic oligonucleotide primer
gactatggtaccatgtatacagtaggagattacctattag

SEQ ID NO: 79 - DNA, 38 bp, artificial; identical to SEQ ID NO: 62
  synthetic oligonucleotide primer

SEQ ID NO: 80 - DNA, 38 bp, artificial; identical to SEQ ID NO: 63
  synthetic oligonucleotide primer

SEQ ID NO: 81 - DNA, 38 bp, artificial; identical to SEQ ID NO: 70
  synthetic oligonucleotide primer

SEQ ID NO: 82 - DNA, 38 bp, artificial; identical to SEQ ID NO: 71
  synthetic oligonucleotide primer

SEQ ID NO: 83 - protein, 29 aa, artificial
  Lactococcus lactis 2-ketoacid decarboxylase (KIVD) active site
QGTSFFGASSDGYTVERIHLKKMGKLFAE

SEQ ID NO: 84 - protein, 30 aa, artificial
  Salmonella typhimurium indolepyruvate decarboxylase (IPDC) active site
QGTAAFGAAADGYTVERAIHLRTVTRALEA

SEQ ID NO: 85 - DNA, 54 bp, artificial
  synthetic oligonucleotide primer
tgcatcgaattcaggagaaattaactatgaacgagtacgcccccctgcgtttgc

SEQ ID NO: 86 - DNA, 36 bp, artificial
  synthetic oligonucleotide primer
tgcatcaagctttcagatatgcaaggcgtggcccag

SEQ ID NO: 87 - DNA, 52 bp, artificial
  synthetic oligonucleotide primer
gggcccaagcttaggagaaattaactatgatgaacttcaacaatgttttccg

SEQ ID NO: 88 - DNA, 38 bp, artificial
  synthetic oligonucleotide primer
gggccctctagattatgagtcatgatttactaaaggct

SEQ ID NO: 89 - DNA, 55 bp, artificial
  synthetic oligonucleotide primer
gggcccaagcttaggagaaattaactatgatgagtcaggcgctaaaaaatttact

SEQ ID NO: 90 - DNA, 38 bp, artificial
  synthetic oligonucleotide primer
gggccctctagattaattgtgattacgcatcacccctt

SEQ ID NO: 91 - protein, 410 aa, Pseudomonas putida
MNEYAPLRLHVPEPTGRPGCQTDFSYLRLNDAGQARKPAIDVDAADTADLSYSLVRVLDEQGDAQGPWAEDIDPQILRQGMRAMLKTRIFDSRMVVAQRQKKMSFYMQSLGEEAIGSGQALALNRTDMCFPTYRQQSILMARDVSLVEMICQLLSNERDPLKGRQLPIMYSVREAGFFTISGNLATQFVQAVGWAMASAIKGDTKIASAWIGDGATAESDFHTALTFAHVYRAPVILNVVNNQWAISTFQAIAGGESTTFAGRGVGCGIASLRVDGNDFVAVYAASRWAAERARRGLGPSLIEWVTYRAGPHSTSDDPSKYRPADDWSHFPLGDPIARLKQHLIKIGHWSEEEHQAVTAELEAAVIAAQKEAEQYGTLANGHIPSAASMFEDVYKEMPDHLRRQRQELGV

SEQ ID NO: 92 - protein, 339 aa, Pseudomonas putida
MATTTMTMIQALRSAMDVMLERDDNVVVYGQDVGYFGGVFRCTEGLQNKYGKSRVFDAPISESGIVGTAVGMGAYGLRPVVEIQFADYFYPASDQIVSELARLRYRSAGEFIAPLTLRMPCGGGIYGGQTHSQSPEAMFTQVCGLRTVMPSNPYDAKGLLIASIECDDPVIFLEPKRLYNGPFDGHHDRPVTPWSKHPHSAVPDGYYTVPLDKAAITRPGNDVTVLTYGTTVYVAQVAAEESGVDAEVIDLRSLWPLDLDTIVESVKKTGRCVVVHEATRTCGFGAELVSLVQEHCFHHLEAPIERVTGWDTPYPHAQEWAYFPGPSRVGAALKKVMEV

SEQ ID NO: 93 - protein, 423 aa, Pseudomonas putida
MGTHVIKMPDIGEGIAQVELVEWFVKVGDIIAEDQVVADVMTDKATVEIPSPVSGKVLALGGQPGEVMAVGSELIRIEVEGSGNHVDVPQPKPVEAPAAPIAAKPEPQKDVKPAVYQAPANHEAAPIVPRQPGDKPLASPAVRKRALDAGIELRYVHGSGPAGRILHEDLDAFMSKPQSNAGQAPDGYAKRTDSEQVPVIGLRRKIAQRMQDAKRRVAHFSYVEEIDVTALEALRQQLNSKHGDSRGKLTLLPFLVRALVVALRDFPQINATYDDEAQIITRHGAVHVGIATQGDNGLMVPVLRHAEAGSLWANAGEISRLANAARNNKASREELSGSTITLTSLGALGGIVSTPVVNTPEVAIVGVNRMVERPVVIDGQIVVRKMMNLSSSFDHRVVDGMDAALFIQAVRGLLEQPACLFVE